(19) United States
(12) Patent Application Publication
(10) Pub. No.: US 2024/0102073 A1
(43) Pub. Date: Mar. 28, 2024

(54) GENETICALLY MODIFIED ORGANISMS FOR THE PRODUCTION OF STEROID DERIVATIVES

(71) Applicant: SANDHILL ONE, LLC, West Palm Beach, FL (US)

(72) Inventors: JAYACHANDRA P. REDDY, West Palm Beach, FL (US); J. GREGORY REID, Wellington, FL (US); RICHARD REUMERMAN, Cambridge (GB); BERNHARD J. PAUL, Arlington, MA (US); URSULA SCHELL, Cambridge (GB)

(21) Appl. No.: 18/009,057

(22) PCT Filed: Jun. 8, 2021
(86) PCT No.: PCT/US21/36287

§ 371 Date: Dec. 8, 2022

Related U.S. Application Data
(60) Provisional application No. 63/037,432, filed on Jun. 10, 2020.
(60) Provisional application No. 63/162,229, filed on Mar. 17, 2021.

(51) Int. Cl. C12P 33/00; C12N 9/02

(52) U.S. Cl. CPC C12P 33/00; C12N 9/001; C12N 9/0077; C12N 9/0095; C12Y 103/99003

(57) ABSTRACT

Provided are biosynthetic processes for producing sterol derivatives, and to non-naturally occurring organisms capable of producing sterol derivatives. More specifically, genetically modified non-naturally occurring organisms for producing KCEA, KCDA, and related compounds, from cholesterol, β-sitosterol, campesterol and their analogs, are provided.

This application hereby incorporates-by-reference a sequence listing submitted herewith in ST.25.txt format, having a file name of WO_SEQUENCE_LISTING_ST25.txt, created on Jun. 31, 2021, which is 49,428 bytes in size.

### FIELD OF THE INVENTION

The present invention relates to biosynthetic processes for producing sterol derivatives, and to non-naturally occurring organisms capable of producing sterol derivatives. More specifically, the invention relates to the use of genetically modified non-naturally occurring organisms to produce KCEA, KCDA, and related compounds, from cholesterol, β-sitosterol and related compounds.

### BACKGROUND OF THE INVENTION

Steroids represent a specific class of terpenoid lipids that contain a gonane core of four fused cycloalkane rings represented by the following chemical structure:

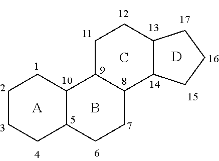

The gonane core can possess one or a plurality of degrees of ethylenic unsaturation; it can be modified by one or more organic functional group moieties; and it can exist in several different optical orientations. The steroid superfamily thus includes various structures such as sterols (e.g., cholesterol, β-sitosterol, ergosterol); bile acids; corticoids; cardiac aglycones; hormones; vitamin D; and insect molting hormones.

Steroids perform multiple functions in living organisms of the animal and plant kingdoms, and structural modifications of steroids highly affect their biological activity. Positions of the hydroxyl groups and stereochemistry around carbons to which they are attached in the cycloalkane rings, as well as in the side chain of steroids, are of critical importance. For example, the presence of hydroxyl functions in positions 11β and 17α is essential for anti-inflammatory activity (e.g., cortisol, prednisolone), the 14β-hydroxyl group is typically found in cardioactive steroids, the 7-hydroxylated derivatives of dehydroepiandrosterone (DHEA) and epiandrosterone (EpiA) have neuroprotective effects, and the 1α- and 25α-hydroxyl functions are of significance for vitamin D₃ activity.

In recent years metabolic engineering has allowed the production of new steroid-producing strains. As reported in the review by Fernández-Cabezón et al. (Frontiers in Microbiology, Volume 9, Article 958) (May 2018), for instance, Galán et al. (Microb. Biotechnol. 10, 138-150) (2017) produced mutant strains of Mycobacterium smegmatis to produce ADD (“1,4-androstadiene-3,17-dione) and AD (“4-androstene-3,17-dione”) sterol derivatives. Yao et al. (Metab. Eng. 24, 181-191) (2014) developed multiple-gene-deletion mutants of Mycobacterium neoaurum to produce 90H-AD and C-22 sterol derivatives. Wei et al. (Appl. Environ. Microbiol. 76, 4578-4582) (2010) engineered an AD-producing mutant by deleting the KstD gene of Mycobacterium neoaurum NwlB-01. Other authors have attempted to construct mutant strains producing steroidal intermediates from sterols in species of the genus Rhodococcus. However, the presence of multiple steroid catabolic pathways and the existence of basal levels of certain isoenzymes (e.g., KstD, KshA, and KshB) have hindered the development of stable producers in these species.

What is needed are new genetically modified microbial organisms having the activity of critical enzymes knocked out, capable of halting steroid mineralization at specific junctures in the degradation pathway, and thereby producing commercially useful steroids and steroid intermediates in high yield with a minimum of unwanted byproducts.

What is especially needed are genetically modified organisms having selective activity of critical enzymes knocked out, disabled, or otherwise disrupted, capable of producing KCEA, KCDA, and similar compounds, from cholesterol, β-sitosterol, campesterol, and other similar sterols having alkyl side chains at least 5 carbons in length.

### SUMMARY OF INVENTION

The inventors have discovered several genetically modified organisms with the unexpected ability to produce KCEA and KCDA and related sterols in high yield from commercially available sterols such as β-sitosterol, cholesterol, and campesterol. The organisms preferably have essential enzymatic activity encoded in the chromosomes of the native organism disrupted as by homologous recombination or induced mutagenesis. By disrupting an organism's 9,10-seco degradation pathway, particularly 9-alpha-hydroxylation activity, and by disrupting the organism's ability to degrade 4-(steroid-17-yl)pentanoyl CoA side chains, particularly at the 22,23-position, valuable sterol derivatives such as KCDA and KCEA can be produced in surprisingly high yield. By further disrupting 3-ketosteroid delta-1 dehydrogenase activity, one is able to produce KCEA in preference to KCDA.

Thus, in a first principal embodiment, the invention provides a non-naturally occurring steroid degrading microbial organism comprising the following enzymatic activities disrupted: aerobic 9,10-seco degradation activity and 4-(steroid-17-yl)pentanoyl side chain degradation activity.

In a second principal embodiment the invention provides a non-naturally occurring steroid degrading microbial organism comprising the following enzymatic activities disrupted: steroid 9-alpha-hydroxylation activity and 4-(steroid-17-yl)pentanoyl side chain degradation activity.

In a third principal embodiment the invention provides a non-naturally occurring steroid degrading microbial organism characterized by the following enzymatic activities disrupted: (a) one or more 3-ketosteroid-9-alpha-hydroxylase isoenzymes disrupted at an oxygenase subunit, a ferredoxin reductase subunit, or a combination thereof, and (b) one or more acyl-CoA dehydrogenase isoenzymes that act on steroid CoA esters having five carbon side chains.

In a fourth principal embodiment the invention provides a non-naturally occurring steroid degrading microbial organism characterized by the following enzymatic activities disrupted: (a) one or more 3-ketosteroid-9-alpha-hydroxylase isoenzymes disrupted at an oxygenase subunit, a ferredoxin reductase subunit, or a combination thereof, (b) one or more acyl-CoA dehydrogenase isoenzymes that act on steroid CoA esters having five carbon side chains; and (c) one or more 3-ketosteroid delta-1 dehydrogenase isoenzymes.

Additional advantages of the invention are set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying drawings, which are incorporated in and constitute part of this specification, illustrate several embodiments of the invention and together with the description serve to explain the principles of the invention.

FIG. 1 is a flowchart diagram showing the order of gene knockouts described in example 1, generating Rhodococcus strains SAND001-SAND006, and the product of these strains when grown on phytosterols or cholesterol.

FIG. 2 is a flowchart diagram showing the order of gene knockouts described in example 2, generating Rhodococcus strains SAND007-SAND012, and the product of these strains when grown on phytosterols or cholesterol.

FIG. 3 is a flowchart diagram showing the order of gene knockouts described in example 4 and example 5, leading to Rhodococcus strains SAND013-SAND015, and the product of these strains when grown on phytosterols or cholesterol.

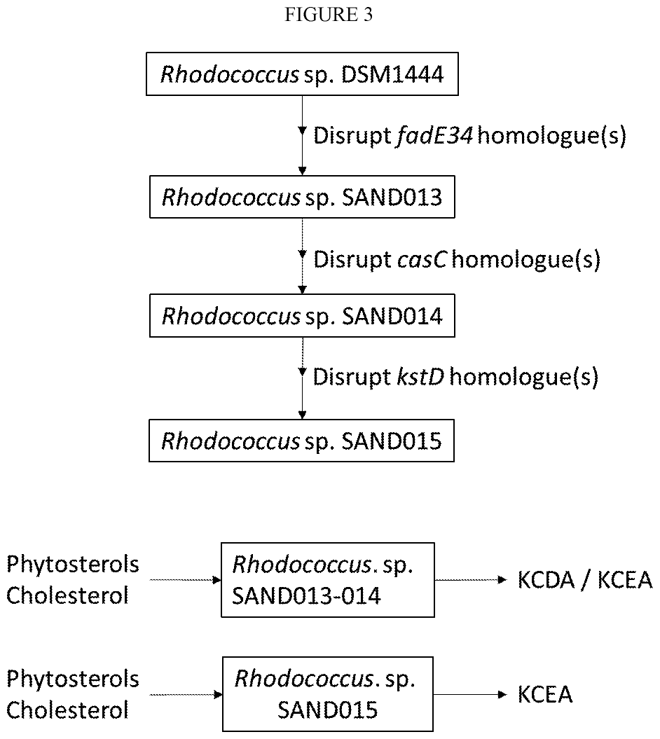

FIG. 4 is a flowchart diagram showing the order of gene knockouts described in example 7 and example 8, generating Mycobacterium strains SAND016-SAND019, and the product of these strains when grown on phytosterols or cholesterol.

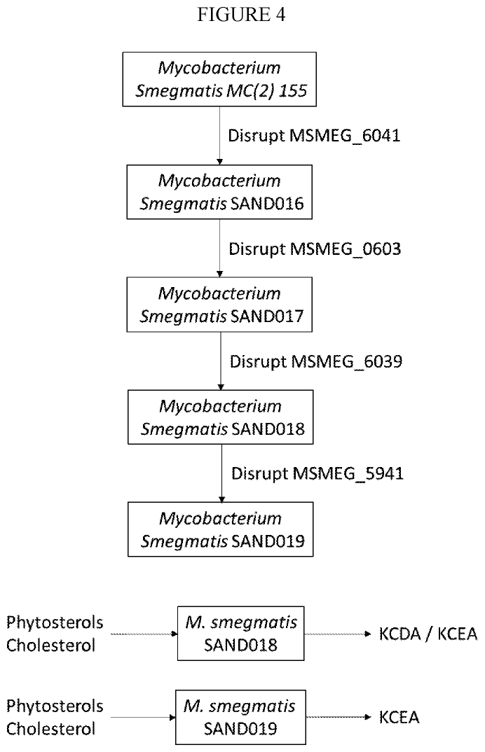

FIG. 5 is a graphical depiction of the resulting HPLC/MS traces from the experiment described in Example 14. A: UV trace of the extracted broth sample, B: Extracted Ion Chromatogram (EIC) for m z 373.55 (KCEA) of the extracted broth sample, C: UV trace of the KCEA standard, TIC of the KCEA standard.

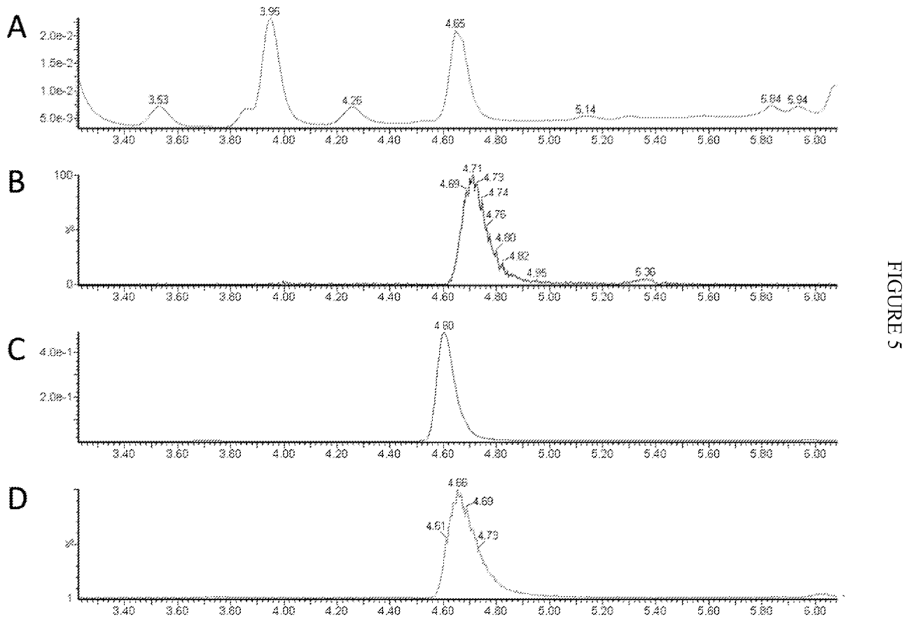

FIG. 6 is an MS spectra comparison of KCEA peaks of the extracted broth sample (A) and the KCEA standard (B) reported in the Examples.

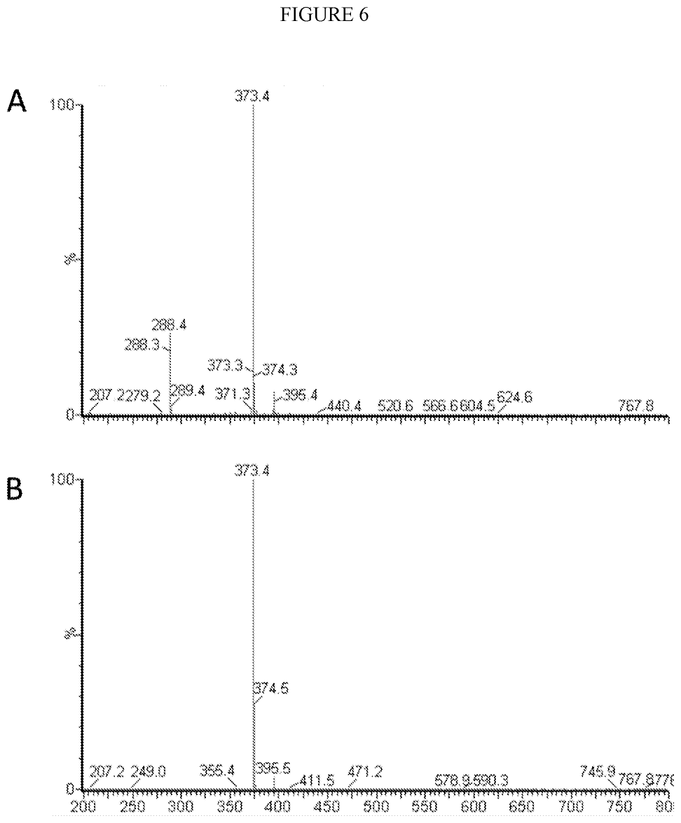

FIG. 7 is an analysis of Mycobacterium neoaurum SAND029 fed with a plant sterol mixture as described in example 21. A) LCMS trace (UV at 246 nm) of an isolated KCEA standard (top) and the whole broth extract (bottom), B) MS spectra of the isolated KCEA standard (top) and of the whole broth extract (bottom).

FIG. 8 is an analysis of Mycobacterium neoaurum SAND030 fed with cholesterol as described in example 23. A) LCMS trace (UV at 246 nm) of an isolated KCEA standard (top) and the whole broth extract (bottom), B) MS spectra of the isolated KCEA standard (top) and the whole broth extract (bottom).

### DETAILED DESCRIPTION

### Definitions and Use of Terms

As used in this specification and in the claims which follow, the singular forms “a,” “an” and “the” include plural referents unless the context clearly dictates otherwise. “Comprise” and variations of the word, such as “comprising” and “comprises,” means “including but not limited to,” and is not intended to exclude, for example, other additives, components, integers or steps. When an element is described as comprising a plurality components, steps or conditions, it will be understood that the element can also be described as comprising any combination of such plurality, or “consisting of” or “consisting essentially of” the plurality or combination of components, steps or conditions.

Alkyl refers to a saturated straight or branched hydrocarbon having from 1 to 20 carbons, optionally substituted by one or more acyl, hydroxy, or carboxylic acids. Alkyl preferably refers simply to a saturated straight or branched hydrocarbon having from 1 to 20 carbons.

Isozymes or isoenzymes are enzymes that differ in amino acid sequence but catalyze the same chemical reaction. Isoenzymes with the same activity can exist within the same microbial species, or across varying microbial species.

The “native state” of an organism refers to the naturally occurring state of an organism prior to its modification according to the methods of the present invention, preferably through recombinant or mutagenetic techniques. Thus, for example, when this document states that “said activities are encoded in the chromosomes of said organism in its native state,” it means that “said activities are encoded in the chromosomes prior to induced modification.”

FadE34 (i.e. ChsE3) stands for the 5-carbon ACAD involved in cholesterol degradation and CasC for the homologue ACAD involved in cholate degradation. Rhodococcus jostii RHA1, Mycobacterium smegmatis, and Mycobacterium neoaurum all code for both FadE34 and CasC.

### DISCUSSION

As mentioned in the summary of the invention, the inventors have discovered mutant microbial organisms derived from organisms having the ability to degrade steroid molecules, that are able to stop the steroid degradation at key enzymatic steps and thereby produce desirable steroid derivatives in high yield with little or no production of unwanted by-products.

Thus, in a first principal embodiment, the invention provides a non-naturally occurring steroid degrading microbial organism comprising the following enzymatic activities disrupted: aerobic 9,10-seco degradation activity and 4-(steroid-17-yl)pentanoyl side chain degradation activity.

In a second principal embodiment the invention provides a non-naturally occurring steroid degrading microbial organism comprising the following enzymatic activities disrupted: steroid 9-alpha-hydroxylation activity and 4-(steroid-17-yl)pentanoyl side chain degradation activity.

In a third principal embodiment the invention provides a non-naturally occurring steroid degrading microbial organism characterized by the following enzymatic activities disrupted: (a) one or more 3-ketosteroid-9-alpha-hydroxylase isoenzymes disrupted at an oxygenase subunit, a ferredoxin reductase subunit, or a combination thereof, and (b) one or more acyl-CoA dehydrogenase isoenzymes that act on steroid CoA esters having five carbon side chains.

In a fourth principal embodiment the invention provides a non-naturally occurring steroid degrading microbial organism characterized by the following enzymatic activities disrupted: (a) one or more 3-ketosteroid-9-alpha-hydroxylase isoenzymes disrupted at an oxygenase subunit, a ferredoxin reductase subunit, or a combination thereof, (b) one or more acyl-CoA dehydrogenase isoenzymes that act on steroid CoA esters having five carbon side chains; and (c) one or more 3-ketosteroid delta-1 dehydrogenase isoenzymes.

The precise enzymatic activity to be disrupted in the genetically modified organism is a function of three separate elements: (i) the desired end product, (ii) the starting material for the production of the end product, and (iii) the native enzymatic activity of the genetically modified organism. Armed with the knowledge of these three elements, the inventors have rationally designed genetically modified organisms capable of producing steroid degradation products in high yields using known industrial techniques for the growth of microorganisms, and the harvesting of and purification of desired end products from those microorganisms. For example, by growing a microbial organism derived from the genera Rhodococcus or Mycobacterium on a sterol such as cholesterol, β-sitosterol, or campesterol, in an appropriate growing environment, it is possible to obtain KCEA or KCDA, or a combination of KCEA and KCDA, in conversion yields exceeding 20%, 35%, or even 50%.

### End Products of the Current Invention

The end product of the processes of the current invention can be broadly defined as steroids comprising:
a) an intact gonane core;
b) a ketone at the 3-position of the A ring; and
c) a 4-yl-pentanoic acid at the 17-position of the D ring, as in KCEA and KCDA. For ease of discussion, this genre of end products will be referred to herein as 4-(3-ketosteroid-17-yl)pentanoic acids.

In more particular embodiments, the 4-(3-ketosteroid-17-yl)pentanoic acids can be characterized by one or a combination of the following modifications to the gonane core:
a) methyl substitution at the 10-carbon on the gonane core;
b) methyl substitution at the 13-carbon on the gonane core; and/or
c) one or two degrees of ethylenic unsaturation on the A-ring.

In still further embodiments the 4-(3-ketosteroid-17-yl)pentanoic acid can be characterized by (r) stereochemistry at the 4-yl-pentanoic acid side chain, which will be the same as in the sterol starting material. In still further embodiments, the 4-(3-ketosteroid-17-yl)pentanoic acid is characterized by (r) stereochemistry at the 17 and 20 positions.

Particularly preferred end products are KCEA (3-ketochol-4-enoic acid) and KCDA (3-ketochola-1,4-dienoic acid) and their corresponding alcohols, as defined by the following chemical structures:

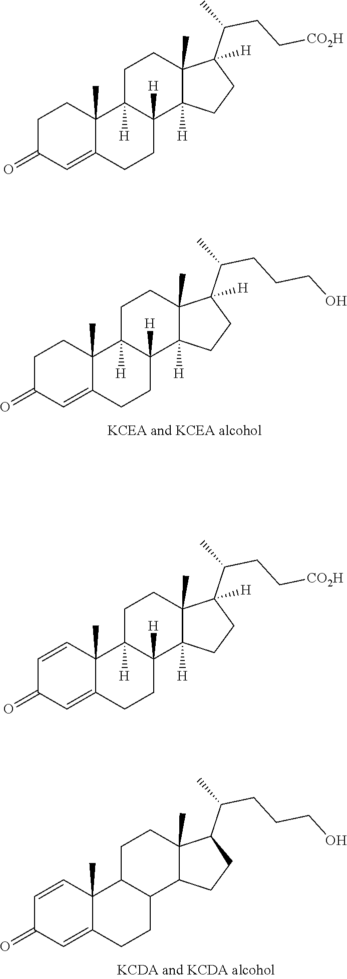

### Starting Materials for the Current Invention

The starting materials for use in the present invention can be broadly defined as any steroid comprising:
a) an intact gonane core;
b) a hydroxy at the 3-position of the A ring; and
c) an alkan-2-yl at the 17-position of the D ring comprising from 6 to 20 carbon atoms. For ease of discussion, this genre of starting materials will be referred to herein as 3-hydroxy, 17-(alkan₍₆₋₂₀₎-2-yl) steroids.

In more particular embodiments, the 3-hydroxy, 17-(alkan₍₆₋₂₀₎-2-yl) steroids will be characterized by:
a) methyl substitution at the 10-carbon on the gonane core;
b) methyl substitution at the 13-carbon on the gonane core; and
c) one degree of ethylenic unsaturation at the 5-6 bond on the B-ring.

In other more particular embodiments, the alkan-2-yl at the 17-position of the D ring will comprise from 6 to 15 carbon atoms, from 7 to 12 carbon atoms, or from 8 to 10 carbon atoms. In preferred embodiments, the alkan-2-yl at the 17-position of the D ring comprises the 8-carbon side chain that characterizes cholesterol, the 9-carbon side chain that characterizes campesterol, or the 10-carbon side chain that characterizes β-sitosterol. Particularly preferred starting materials include cholesterol, β-sitosterol, and campesterol, defined by the following chemical structures:

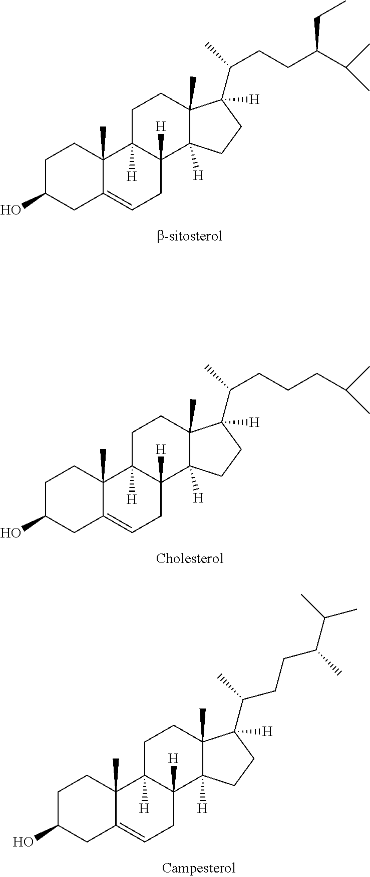

### Steroid Degrading Microbial Organisms

Complete mineralization of the foregoing starting materials in most native organisms capable of degrading steroids typically depends on multiple enzymes with varying activities, and multiple isoenzymes in the same organism often sharing common enzymatic activity. These enzymes can generally be grouped into four separate categories: (1) those involved in A ring degradation, (2) those involved in side chain degradation, (3) those involved in AB ring degradation, and (4) those involved in CD ring degradation. Representative enzymatic activities from various microbial taxa include the following enzymes and their isoenzymes from actinobacteria (with abbreviations from exemplary species given in parentheses):

A Ring Degradation Enzymes
Cholesterol oxidase isoenzymes (ChOx);
3-ketosteroid-delta-1 dehydrogenase isoenzymes (“KstD”);

Side Chain Degradation Enzymes
acyl-CoA dehydrogenase (“ACAD”) isoenzymes that act on steroid CoA esters having five carbon side chains (FadE34 ChsE3 or CasC);
acyl-CoA dehydrogenase (“ACAD”) isoenzymes that act on steroid CoA esters other than steroid CoA esters having five carbon side chains;

AB Ring Degradation Enzymes
3-ketosteroid-9-alpha-hydroxylase isoenzymes, including the subunits responsible for oxygenase activity (“KshA”) and ferredoxin reductase activity (“KshB”);

CD Ring Degradation Enzymes
3-hydroxy-9,10-seconandrost-1,3,5(10)-triene-9,17-dione-4-hydroxylase (oxygenase) (“HsaA”) isoenzymes;
3,4-dihydroxy-9,10-secoandrosta-1,3,5(10)-triene-9,17-dione-4,5-dioxygenase (“HsaC”) isoenzymes;
4,5-9,10-diseco-3-hydroxy-5,9,17-trioxoandrosta-1(10),2-diene-4-oate hydrolase (“HsaD”) isoenzymes;
2-hydroxyhexa-2,4-dienoate hydratase (“HsaE”) isoenzymes;
4-hydroxy-2-oxohexanoate aldolase (“HsaF”) isoenzymes; and
propanol dehydrogenase (“HsaG”) isoenzymes.

For purposes of this invention, any steroid degrading microbial organism comprising in its native state the “essential disruption enzymes” described herein and the “essential retention enzymes” described herein will be suitable for practicing the methods of the current invention. Essential disruption enzymes are steroid degrading enzymes that are present in the native organisms of the present invention whose activity must be disrupted to practice the methods of the current invention.

Essential disruption enzymes thus include acyl-CoA dehydrogenase isoenzymes that act on steroid CoA esters having five carbon side chains (e.g. FadE34 ChsE3 or CasC). In a particularly preferred embodiment, the organism has enzymatic activity responsible for 4-(steroid-17-yl)pentanoyl side chain degradation disrupted. It will be understood that the 4-(steroid-17-yl)pentanoyl side chain can accumulate in the organism as a CoA ester, a carboxylic acid, or an alcohol, and that the exact structure of the side chain is not important to the invention as long as the acyl-CoA dehydrogenase (“ACAD”) isoenzyme responsible for its degradation is disrupted.

Another essential enzymatic activity to have disrupted is the enzymatic activity associated with the two component 3-ketosteroid-9-alpha-hydroxylase (“Ksh”) enzyme system. The Ksh enzyme system includes oxygenase (“KshA”) activity and ferredoxin reductase (“KshB”) activity, and the invention can be practiced by disrupting either activity or both, and one or more isoenzymes responsible for such activities. Put another way, the invention can be practiced by disrupting the 3-ketosteroid-9-alpha-hydroxylase, oxygenase subunit, and/or the 3-ketosteroid-9-alpha-hydroxylase, ferredoxin reductase subunit, of the Ksh enzyme system.

Organisms in which the above two activities are disrupted are capable of producing KCEA and KCDA from an appropriate steroid starting material. In embodiments where a double bond at the C₁₋₂ position in the A ring is not desired, it is also essential to disrupt 3-ketosteroid-delta-1 dehydrogenase isoenzyme (“KstD”) activity as a third class. It is thus possible to produce KCEA in preference to KCDA by also disrupting KstD activity.

It will be understood that the native organisms of the present invention can comprise one or a plurality of isoenzymes responsible for each of the foregoing activities, and that the activity of one or any combination of isoenzymes present in the native microorganism can be disrupted in the practice of the current invention. It will also be understood that the activity disrupted according to the present invention is encoded in the chromosomes of the native organism. Thus, preferred organisms of the present invention are modified by genetic knock-out of the relevant activity through homologous recombination or induced mutagenesis.

It should be noted that preferred organisms for practicing the current invention have just one chromosome but that other suitable organisms contain multiple chromosomes. The term “chromosomes” is not meant to limit the organism to organisms with plural chromosomes, but simply refers to the fact that organisms with single and plural chromosomes are intended by the current invention unless expressly stated to the contrary.

Essential retention enzymes are steroid degrading enzymes that are present in the native organisms of the present invention whose activity must be retained to practice the methods of the current invention. For purposes of this invention essential retention enzymes include one or more acyl-CoA dehydrogenase (“ACAD”) isoenzymes that act on steroid CoA esters other than steroid CoA esters having five carbon side chains, for example, enzymes and isoenzymes necessary for conversion of naturally occurring C8 to C10 side chains to a C5 side chain with a CoA ester or a carboxylic acid at the terminal 24-position of the side chain. Once again, it is not important whether the organism accumulates the CoA ester, a carboxylic acid, or an alcohol of the side chain.

Other essential retention enzymes include enzyme(s) capable of converting a 3-beta-hydroxy-5-ene steroid to a 3-keto-4-ene steroid.

Mutant steroid degrading microbial organisms useful for practicing the methods of the current invention are derived from native organisms that possess the essential disruption enzymes and essential retention enzymes as defined herein, in which the activity of the essential disruption enzymes has been removed, disrupted, or degraded, as through homologous recombination or induced mutagenesis.

Thus, in one embodiment, the mutant organism for practicing the current invention refers to an organism having impaired or degraded activity by the following isoenzymes:
an acyl-CoA dehydrogenase isoenzyme that acts on steroid CoA esters having five carbon side chains (e.g. FadE34 ChsE3 or CasC; an isoenzyme of the Ksh enzyme system having activity disrupted at the 3-ketosteroid-9-alpha hydroxylase, oxygenase subunit, and/or the 3-ketosteroid-9-alpha hydroxylase, ferredoxin reductase subunit; and, optionally, a 3-ketosteroid-delta-1 dehydrogenase isoenzyme (“KstD”); in combination with one or more intact acyl-CoA dehydrogenase (“ACAD”) isoenzymes that do not act on steroid CoA esters having five carbon side chains and one or more enzyme(s) capable of converting a 3-beta-hydroxy-5-ene steroid to a 3-keto-4-ene steroid.

Preferred organisms include Actinobacteria spp. as well as alpha-, beta-, and gamma proteobacteria, all as described by Bergstrand et al. (mBio Volume 7 Issue 2 e00166-16) (American Society of Microbiology) (2016). Actinobacteria preferably include genera in the suborder Corynebacterineae (Amycolicicoccus, Dietzia, Gordonia, Mycobacterium, Nocardia, Rhodococcus, and Tsukamurella) as well as the genera Actinoplanes, Aeromicrobium, Amycolatopsis, Arthrobacter, Nocardioides, Saccharomonospora, Salinispora, Streptomyces, and Thermomonospora. Proteobacteria organisms preferably include individual species within the genera Burkholderia, Comamonas, Cupriavidus, Glaciecola, Hydrocarboniphaga, Marinobacterium, Novosphingobium, Pseudoalteromonas, Pseudomonas, Shewanella, and Sphingomonas.

Exemplary organisms for practicing the current invention include microbial organisms in the genera Rhodococcus or Mycobacterium, for example Rhodococcus jostii RHA1, Rhodococcus sp. DSM 1444 or DSM 1445, Mycobacterium smegmatis MC(2) 155, and Mycobacterium neoaurum NRRL B-3805. Mycobacterium neoaurum NRRL B-3805 is preferred along with other Mycobacterium strains that lacks a gene cluster (the so-called C-19+ cluster described in Mycobacterium smegmatis) that codes for several isoenzymes of KstD, Ksh, etc. (Mycobacterium smegmatis enzyme names: KstD2, KstD3, KshA2, KshB2, HsaA2, HsaC2 and HsaD2. See Fernández-Cabezón et al. (Environ Microbiol. 2018 May; 20(5):1815-1827.). The genes in Mycobacterium neoaurum NRRL B-3805 were not disrupted by homologous recombination; rather, their activity was disrupted by induced mutagenesis and selection for improved AD production. See Fernández-Cabezón et al. (Front. Microbiol., 15 May 2018 https://doi.org/10.3389/fmicb.2018.00958) and Lorraine and Smith (Methods Mol Biol. 2017; 1645:93-108. doi: 10.1007/978-1-4939-7183-1_7).

### Disruption of Enzyme Activity

While any known genetic technique can be used to alter the expression of the targeted isoenzymes, a preferred method involves insertion of a cloning vector, such as a plasmid, bacteriophage (such as phage λ), cosmid, or bacterial artificial chromosome (BAC), which is subsequently replicated and becomes integral to the chromosomal genetic machinery of the host organism.

A preferred plasmid for strain manipulation via homologous recombination or induced mutagenesis is typically based on a common Escherichia coli cloning vector. Entry of the plasmid into the organism would commonly be facilitated by transformation (e.g. chemically or electroporation) or conjugation. Suitable plasmids can be designed and constructed using well-known biotechnology techniques, generally involving the insertion or removal of a DNA fragment to or from the plasmid. One of the earliest commonly used cloning vectors is the pBR322 plasmid. Other cloning vectors include the pUC series of plasmids, although a large number of different cloning plasmid vectors are now available. pK18mobsacB is an exemplary cloning vector that allows mobilization into a wide range of Gram− and Gram+ bacteria.

A preferred method involves the introduction of gene deletions by homologous recombination. In this method, a DNA fragment which comprises the flanking sequences of the area to be deleted is transferred into the desired strain and incorporated by two recombination events or crossover events into the chromosome of the desired strain, or the sequence of a gene present in the relevant strain is exchanged for a gene with deletion or deleted completely. The DNA fragment is in this method typically present in a vector, in particular a plasmid, which preferably cannot be replicated in the strain to be provided with the deletion. In general, a bacterium of the genus Escherichia, preferably of the species Escherichia coli, may be used as auxiliary or intermediate host in which the vector may be replicated.

Examples of such plasmid vectors are the pK mob and pK*mobsacB vectors such as, for example, pK18mobsacB, which are described by Schafer et al. (Gene 145, 69-73 (1994)). These are replicative in Escherichia coli but not for example in Rhodococcus or Mycobacterium. They may be introduced into the target organism by either transformation, or by conjugation due to the presence of mob genes. Particularly suitable vectors are those comprising a gene with a conditionally negatively dominant effect such as, for example, the sacB gene (levansucrase gene) of, for example, Bacillus. In case of pK18mobsacB, the second crossover event can be facilitated by selection on a medium containing 10% sucrose, as described in greater detail in the Examples hereof.

Homologous recombination occurring in a first crossover event which brings about integration, and of a suitable second crossover event which brings about an excision in the target gene or in the target sequence achieves incorporation of the deletion and results in a recombinant bacterium. The gene in which the desired deletion is to take place is referred to as a target gene.

Preferred essential disruption enzymes in Rhodococcus jostii RHA1, for example, are encoded by RHA1_RS21845 (encoding an acyl-CoA dehydrogenase), RHA1_RS28395 (encoding an acyl-CoA dehydrogenase), RHA1_RS22120 (encoding a 3-ketosteroid-9-alpha-hydroxylase subunit A), RHA1_RS12175 (encoding a 3-ketosteroid-9-alpha-hydroxylase subunit A), RHA1_RS28370 (encoding a 3-ketosteroid-9-alpha-hydroxylase subunit A), RHA1_RS40090 (encoding a 3-ketosteroid-9-alpha-hydroxylase subunit A), RHA1_RS22090 (encoding 3-ketosteroid-delta-1-dehydrogenase (KstD)), RHA1_RS12140 (encoding a FAD-binding protein (KstD2)), RHA1_RS28305 (encoding a FAD-binding protein (KstD3)), RHA1_RS28380 (encoding a FAD-binding protein (KstD3b)), RHA1_RS40180 (encoding a 3-ketosteroid-delta-1-dehydrogenase (KstD4)), and RHA1_RS40260 (encoding a 3-ketosteroid-delta-1-dehydrogenase (KstD4b)).

In one embodiment, the organism is Mycobacterium and one or more KstD isoenzymes is disrupted. I.e., the Mycobacterium comprises disrupted KstD enzymatic activity. Particularly preferred is Mycobacterium neoaurum, Mycobacterium neoaurum NRRL B-3805 or Mycobacterium smegmatis MC(2) 155.

In another embodiment, the organism is Mycobacterium and one or more Ksh isoenzymes is disrupted. I.e., the Mycobacterium comprises disrupted Ksh enzymatic activity. Particularly preferred is Mycobacterium neoaurum, Mycobacterium neoaurum NRRL B-3805 or Mycobacterium smegmatis MC(2) 155.

In another embodiment, the organism is Mycobacterium and one or more KstD isoenzymes and one or more Ksh isoenzymes is disrupted. I.e., the Mycobacterium comprises disrupted KstD activity and disrupted Ksh enzymatic activity. Particularly preferred is Mycobacterium neoaurum, Mycobacterium neoaurum NRRL B-3805 or Mycobacterium smegmatis MC(2) 155.

In another embodiment, the organism is Mycobacterium and the one or more acyl-CoA dehydrogenase isoenzymes are selected from the group consisting of ChsE1, ChsE2, ChsE3, and CasC isoenzymes and combinations thereof. Particularly preferred is Mycobacterium neoaurum, Mycobacterium neoaurum NRRL B-3805 or Mycobacterium smegmatis MC(2) 155.

In another embodiment, the organism is Mycobacterium and the one or more acyl-CoA dehydrogenase isoenzymes comprise a combination of ChsE1, ChsE2, ChsE3, and CasC isoenzymes. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes are selected from the group consisting of ChsE1, ChsE2, ChsE3, and CasC isoenzymes and combinations thereof, and one or more KstD isoenzymes is disrupted. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes are selected from the group consisting of ChsE1, ChsE2, ChsE3, and CasC isoenzymes and combinations thereof, and one or more Ksh isoenzymes is disrupted. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes are selected from the group consisting of ChsE1, ChsE2, ChsE3, and CasC isoenzymes and combinations thereof, and one or more Ksh isoenzymes is disrupted. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes comprise the combination of ChsE1, ChsE2, ChsE3, and CasC isoenzymes and combinations thereof, and one or more KstD isoenzymes is disrupted. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes comprise the combination of ChsE1, ChsE2, ChsE3, and CasC isoenzymes and combinations thereof, and one or more Ksh isoenzymes is disrupted. Particularly preferred is Mycobacterium neoaurum, Mycobacterium neoaurum NRRL B-3805 or Mycobacterium smegmatis MC(2) 155.

In one embodiment, the organism is Mycobacterium, the one or more acyl-CoA dehydrogenase isoenzymes comprise the combination of ChsE1, ChsE2, ChsE3, and CasC isoenzymes and combinations thereof, and one or more Ksh isoenzymes is disrupted. Particularly preferred is Mycobacterium neoaurum, Mycobacterium neoaurum NRRL B-3805 or Mycobacterium smegmatis MC(2) 155.

In one particular embodiment of Mycobacterium smegmatis MC(2) 155, preferred essential disruption enzymes are encoded by MSMEG_6041 (acyl-CoA dehydrogenase), MSMEG_0603 (acyl-CoA dehydrogenase), MSMEG_6039 (ferredoxin-NADP reductase), or MSMEG_5941 (3-ketosteroid-delta-1-dehydrogenase), or a combination thereof.

In one particular embodiment of Mycobacterium neoaurum or Mycobacterium neoaurum NRRL B-3805, preferred essential disruption enzymes are encoded by MyAD_RS24250; MyAD_RS03655; MyAD_RS24025; MyAD_RS24020; or a combination thereof.

### Methods of Making Steroids

The invention further relates to methods of making steroids, especially KCEA, KCDA, in addition to UDCA and other downstream products, using the novel organisms of the current invention. Thus, in one embodiment the invention provides a method of making a steroid comprising growing the non-naturally occurring microorganism of any of claims 1-25 on one or more phytosterols to produce KCEA and/or KCDA.

In another embodiment the invention provides a method of making a steroid comprising growing the non-naturally occurring microorganism of any of claims 1-25 on β-sitosterol, campesterol, cholesterol, or a mixture thereof to produce KCEA and/or KCDA.

In still another embodiment the invention provides a method of making a steroid comprising growing the non-naturally occurring microorganism of any of claims 1-25 on β-sitosterol, campesterol, cholesterol, or a mixture thereof to produce KCEA and/or KCDA at a yield greater than 20%, 30%, 40%, 50%, 60%, 70%, or 80%.

Other growth media can be added to support the growth, but a particularly notable additive is cyclodextrin, as described further in the examples.

In any of the foregoing method embodiments, KCEA is preferably produced at a ratio to AD of greater than 0.1:1, 0.2:1, 0.3:1. 0.4:1, 0.5:1, or 0.6:1. Likewise, in any of the foregoing method embodiments, KCEA is preferably produced at a ratio to other major catabolic products (i.e. AD, BA, and KCEA-alcohol) greater than 0.1:1, 0.2:1, 0.3:1. 0.4:1, or 0.5:1.

In any of the foregoing method embodiments, the method may optionally further comprise converting said KCEA or KCDA to UDCA. KCEA, KCDA, or a mixture of the two compounds may be converted to UDCA using various prior art methods including the following three step conversion. First, the double bond(s) of the A-ring are hydrogenated to preferentially give 3-keto-5β-cholanic acid. See Tsuji Natsuko, et al, Journal of Organic Chemistry, 1980, vol. 45, p. 2729 (showing high selectivity for the 5f-product starting with both 3-keto-4-ene steroids and 3-keto-1,4-diene steroids). The 3-keto-5β-cholanic acid can then be treated with sodium borohydride in aqueous THE to provide lithocholic acid. See CN 112375117 (2021) (Exemplify the reaction using sodium borohydride in aqueous THF.) The lithocholic acid can then be converted through 7β-Hydroxylation to give UDCA via bioconversion using Fusarium equiseti M-41. See U.S. Pat. No. 4,579,819 (1986) (exemplifying same reaction using Fusarium equiseti M-41).

Thus, the conversion to UDCA can comprise the following steps: (a) catalytic hydrogenation of KCDA or KCEA to 3-keto-5β-cholanic acid, (b) reduction of the 3-ketone of 3-keto-5β-cholanic acid to lithocholic acid by treatment with sodium borohydride in aqueous THF, and (c) conversion of lithocholic acid to ursodeoxycholic acid by enzymatic 7-beta-hydroxylation by exposure to Fusarium equiseti M-41.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated and are intended to be purely exemplary of the invention and are not intended to limit the scope of what the inventors regard as their invention.

### General Methods

Isolation, handling and manipulation of DNA are carried out using standard methods (Green and Sambrook, 2012), which includes digestion with restriction enzymes, PCR, cloning techniques and transformation of bacterial cells.

Genomic DNA for sequencing is isolated using established methods such as the salting out method (Pospiech and Neumann, 1995). For PCR-ready genomic DNA prep FastDNA Spin Kit for Soil (MP Biomedicals) is used following the manufacturer's protocol.

### Media

Media are made up as follows, unless otherwise indicated. To make 1 liter, all ingredients are dissolved in 800 mL deionized water in the order indicated. Deionized water is then added to a final volume of 1 liter, after which the liquid is sterilized by autoclaving at 121° C. for 20 minutes.

Lysogeny broth (LB) contains 10 g/L tryptone, 10 g/L NaCl and 5 g/L yeast extract. To make LB agar plates, 15 g/L agar is added prior to sterilization.

Trypticase soy broth (TSB) is sourced as a pre-mixed powder from Sigma-Aldrich (cat no. 22092) and contains 17 g/L tryptone, 3 g/L soytone, 2.5 g/L glucose, 5 g/L sodium chloride and 2.5 g/L dipotassium phosphate. It is prepared according to the manufacturer's instructions.

Trypticase soy agar (TSA), is sourced as a pre-mixed powder from Sigma-Aldrich (cat no. 22091) and contains 17 g/L tryptone, 3 g/L soytone, 2.5 g/L glucose, 5 g/L sodium chloride, 2.5 g/L dipotassium phosphate and 15 g/L agar. It is prepared according to the manufacturer's instructions.

Middlebrook 7H9 broth is sourced as a pre-mixed powder from Sigma-Aldrich (cat no. M0178) and made up following the manufacturer's instructions. 0.05% Tween 80 is added prior to sterilization and ADC growth supplement (Sigma-Aldrich cat no. M0553) is added to make complex Middlebrook 7H9. To make Middlebrook 7H9 minimal media, ADC growth supplement is omitted, and 14 mM glycerol is substituted for Tween 80 in the recipe above or as indicated in the examples.

M3 media contains 0.5 g/L KH₂PO₄, 0.5 g/L K₂HPO₄, 1.5 g/L (NH₄)₂HPO₄, 0.005 g/L FeSO₄·7H₂O, 0.002 g/L ZnSO₄·7H₂O, 0.2 g/L MgSO₄·7H₂O, 5 g/L yeast extract and 5 g/L glycerol.

M3-Tw medium is as M3 but supplemented with 0.05% (v/v) filter sterilised Tween 80,

M3 and M3-Tw agar plates are prepared as above but with 18 g/L agar.

Medium A contains 8 g/L Difco nutrient broth and 1 g/L yeast extract. The pH is set to 7.0 prior to sterilization.

Mycobacterium minimal bioconversion medium contains 0.5 g/L urea, 3 g/L (NH₄)₂PO₄, 4 g/L KH₂PO₄, 1 g/K K₂HPO₄, 0.2 g/L MgSO₄ 7H₂O, 0.01 g/L FeSO₄ 7H₂O, 0.002 g/L ZnSO₄ 7H₂O. The pH is set to 7.0 prior to sterilization.

### Materials

Restriction enzymes are purchased from New England Biolabs (NEB) or Promega. Media components, chemicals and PCR primers are obtained from Sigma-Aldrich (Merck).

### Deletion Mutagenesis

Unmarked gene deletion mutagenesis is used to delete all genes. In brief, upstream homology sequences and downstream homology sequences are amplified by PCR from a genomic DNA template using standard methods. Care should be taken to use genomic DNA from the correct strain, as the flanking homology sequences of certain target genes overlap with previously disrupted target genes. Plasmid pK18mobsacB (ATCC 87097) is linearized by restriction digest and the PCR products are inserted using the InFusion cloning kit (Takara Bio.) following the manufacturer's instructions. Insertion of the homology sequences is confirmed by restriction digest and sequencing. The resulting plasmid is introduced into Rhodococcus jostii, Rhodococcus sp. DSM1444 or Mycobacterium smegmatis or Mycobacterium neoaurum NRRL B-3805 by electroporation following standard methods (Bibb and Hatfull, 2002; van der Geize et al., 2000; Lorraine and Smith, 2017). First, cross-over colonies are selected for using kanamycin resistance and sensitivity to 10% sucrose (Geize et al., 2001; Pelicic et al., 1996), secondly secondary recombinant colonies are selected for using kanamycin sensitivity and sucrose tolerance.

Deletion mutants are checked by PCR and sequencing using primers as indicated in the examples. Gene IDs used in the examples are taken from the published genome sequences available from NCBI (https://www.ncbi.nlm.nih.gov/): Accession number NC_008268 for Rhodococcus jostii RHA1 and accession number NC_008596 for Mycobacterium smegmatis mc(2) 155 and accession number NZ_CP011022 for Mycobacterium neoaurum NRRL B-3805. Gene names are those used either in the genome sequences or in literature.

### Whole Genome Sequencing

Bacterial genomes are sequenced using Illumina sequencing with hiSeq PCR-free paired-end data and mate pair. Assembly, annotation and analysis are performed using standard software, such as Biopython, BLAST, Artemis, ACT, Mauve, Phyre 2 and Interpro.

### Analysis of Culture Extracts

Following solvent extraction of liquid cultures as described in the Examples, the samples were analyzed for production of KCEA and KCDA on an Agilent 1100 HPLC with a Waters XSelect CSH C18 column, (2.1 mm×50 mm×3.5 m) fitted with a Waters VanGuard and an Acquity in line column filter and operated at 60° C. The mobile phase consisted of solvent A (0.005 M ammonium acetate, 0.012% formic acid) and solvent B (95% methanol, 5% water, 0.012% formic acid) with a flow rate of 1.0 mL/minute. A gradient was run from 50% solvent B to 100% solvent B over 9.5 minutes, Samples were analyzed by UV at 204 nm and 246 nm and by MS using a Waters ZQ single quadrupole MS running in electrospray positive ion mode with a mass range m/z of 200-800).

### Example 1. Inactivation of FadE34 ChsE3, CasC, KshA Homologues in Rhodococcus jostii RHA1

Plasmid pSAND001, to generate a ΔFadE34 (RHA1_RS21845) mutant, is constructed as follows. Plasmid pK18mobsacB is cleaved with restriction enzymes BamHI and PstI. The upstream homology sequence is amplified by PCR using genomic DNA as template using primer pair SEQ ID NO. 1 and SEQ ID NO. 2 (to generate a product of 1525 bp). The downstream homology sequence is amplified by PCR using genomic DNA as template using primer pair SEQ ID NO. 3 and SEQ ID NO. 4 (to generate a product of 1530 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 6, SEQ ID NO. 7 and SEQ ID NO. 8.

Rhodococcus jostii RHA1 is transformed with plasmid pSAND001 by electroporation, using standard methods, after which the cell suspension is spread onto LB agar plates with kanamycin. Single colonies are then transferred to LB agar plates containing 10% sucrose. Single colonies are checked for successful disruption of FadE34 by PCR using primer pair SEQ ID NO. 6 and SEQ ID NO. 7, where presence of a 1407 bp PCR product and absence of a 3465 bp PCR product indicates successful disruption. The resulting strain is named Rhodococcus jostii SAND001.

Plasmid pSAND002, to generate a ΔCasC (RHA1_RS28395) mutant, is constructed as follows. Plasmid pK18mobsacB is cleaved with restriction enzymes BamHI and PstI. The upstream homology sequence is amplified by PCR using genomic DNA as template using primer pair SEQ ID NO. 9 and SEQ ID NO. 10 (to generate a product of 1528 bp). The downstream homology sequence is amplified by PCR using genomic DNA as template using primer pair SEQ ID NO. 11 and SEQ ID NO. 12 (to generate a product of 1530 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 13, SEQ ID NO. 14 and SEQ ID NO. 8.

Rhodococcus jostii SAND001, described above, is transformed with plasmid pSAND002 by electroporation, using standard methods, after which the cell suspension is spread onto LB agar plates with kanamycin. Single colonies are then transferred to LB agar plates containing 10% sucrose. Single colonies are checked for successful disruption of CasC by PCR using primer pair SEQ ID NO. 13 and SEQ ID NO. 14, where presence of a 1376 bp PCR product and absence of a 2828 bp PCR product indicates successful disruption. The resulting strain is named Rhodococcus jostii SAND002.

Plasmid pSAND003, to generate a ΔKshA1 (RHA1_RS22120) mutant, is constructed as follows. Plasmid pK18mobsacB is cleaved with restriction enzymes BamHI and PstI. The upstream homology sequence is amplified by PCR using genomic DNA as template using primer pair SEQ ID NO. 15 and SEQ ID NO. 16 (to generate a product of 1534 bp). The downstream homology sequence is amplified by PCR using genomic DNA as template using primer pair SEQ ID NO. 17 and SEQ ID NO. 18 (to generate a product of 1568 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 19, SEQ ID NO. 20 and SEQ ID NO. 8.

Rhodococcus jostii SAND002, described above, is transformed with plasmid pSAND003 by electroporation, using standard methods, after which the cell suspension is spread onto LB agar plates with kanamycin. Single colonies are then transferred to LB agar plates containing 10% sucrose. Single colonies are checked for successful disruption of KshA1 by PCR using primer pair SEQ ID NO. 19 and SEQ ID NO. 20, where presence of a 1422 bp PCR product and absence of a 2490 bp PCR product indicates successful disruption. The resulting strain is named Rhodococcus jostii SAND003.

Plasmid pSAND004, to generate a ΔKshA2 (RHA1_RS12175) mutant, is constructed as follows. Plasmid pK18mobsacB is cleaved with restriction enzymes BamHI and PstI. The upstream homology sequence is amplified by PCR using genomic DNA as template using primer pair SEQ ID NO. 21 and SEQ ID NO. 22 (to generate a product of 1530 bp). The downstream homology sequence is amplified by PCR using genomic DNA as template using primer pair SEQ ID NO. 23 and SEQ ID NO. 24 (to generate a product of 1528 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 25, SEQ ID NO. 26 and SEQ ID NO. 8.

Rhodococcus jostii SAND003, described above, is transformed with plasmid pSAND004 by electroporation, using standard methods, after which the cell suspension is spread onto LB agar plates with kanamycin. Single colonies are then transferred to LB agar plates containing 10% sucrose. Single colonies are checked for successful disruption of KshA2 by PCR using primer pair SEQ ID NO. 25 and SEQ ID NO. 26, where presence of a 1380 bp PCR product and absence of a 2436 bp PCR product indicates successful disruption. The resulting strain is named Rhodococcus jostii SAND004.

Plasmid pSAND005, to generate a ΔKshA3 (RHA1_RS28370) mutant, is constructed as follows. Plasmid pK18mobsacB is cleaved with restriction enzymes BamHI and PstI. The upstream homology sequence is amplified by PCR using genomic DNA as template using primer pair SEQ ID NO. 27 and SEQ ID NO. 28 (to generate a product of 1526 bp). The downstream homology sequence is amplified by PCR using genomic DNA as template using primer pair SEQ ID NO. 29 and SEQ ID NO. 30 (to generate a product of 1532 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 31, SEQ ID NO. 32 and SEQ ID NO. 8.

Rhodococcus jostii SAND004, described above, is transformed with plasmid pSAND005 by electroporation, using standard methods, after which the cell suspension is spread onto LB agar plates with kanamycin. Single colonies are then transferred to LB agar plates containing 10% sucrose. Single colonies are checked for successful disruption of KshA3 by PCR using primer pair SEQ ID NO. 31 and SEQ ID NO. 32, where presence of a 1374 bp PCR product and absence of a 2427 bp PCR product indicates successful disruption. The resulting strain is named Rhodococcus jostii SAND005.

Plasmid pSAND006, to generate a ΔKshA4 (RHA1_RS40090) mutant, is constructed as follows. Plasmid pK18mobsacB is cleaved with restriction enzymes BamHI and PstI. The upstream homology sequence is amplified by PCR using genomic DNA as template using primer pair SEQ ID NO. 33 and SEQ ID NO. 34 (to generate a product of 1535 bp). The downstream homology sequence is amplified by PCR using genomic DNA as template using primer pair SEQ ID NO. 35 and SEQ ID NO. 36 (to generate a product of 1532 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 37, SEQ ID NO. 38 and SEQ ID NO. 8.

Rhodococcus jostii SAND005, described above, is transformed with plasmid pSAND006 by electroporation, using standard methods, after which the cell suspension is spread onto LB agar plates with kanamycin. Single colonies are then transferred to LB agar plates containing 10% sucrose. Single colonies are checked for successful disruption of KshA4 by PCR using primer pair SEQ ID NO. 37 and SEQ ID NO. 38, where presence of a 1401 bp PCR product and absence of a 2457 bp PCR product indicates successful disruption. The resulting strain is named Rhodococcus jostii SAND006.

### Example 2. Inactivation of KstD Homologues in Rhodococcus jostii SAND006

Plasmid pSAND007, to generate a ΔKstD (RHA1_RS22090) mutant, is constructed as follows. Plasmid pK18mobsacB is cleaved with restriction enzymes BamHI and PstI. The upstream homology sequence is amplified by PCR using genomic DNA as template using primer pair SEQ ID NO. 39 and SEQ ID NO. 40 (to generate a product of 1533 bp). The downstream homology sequence is amplified by PCR using genomic DNA as template using primer pair SEQ ID NO. 41 and SEQ ID NO. 42 (to generate a product of 1528 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 43, SEQ ID NO. 44 and SEQ ID NO. 8.

Rhodococcus jostii SAND006, described above, is transformed with plasmid pSAND007 by electroporation, using standard methods, after which the cell suspension is spread onto LB agar plates with kanamycin. Single colonies are then transferred to LB agar plates containing 10% sucrose. Single colonies are checked for successful disruption of KstD by PCR using primer pair SEQ ID NO. 43 and SEQ ID NO. 44, where presence of a 1387 bp PCR product and absence of a 2851 bp PCR product indicates successful disruption. The resulting strain is named Rhodococcus jostii SAND007.

Plasmid pSAND008, to generate a ΔKstD2 (RHA1_RS12140) mutant, is constructed as follows. Plasmid pK18mobsacB is cleaved with restriction enzymes BamHI and PstI. The upstream homology sequence is amplified by PCR using genomic DNA as template using primer pair SEQ ID NO. 45 and SEQ ID NO. 46 (to generate a product of 1527 bp). The downstream homology sequence is amplified by PCR using genomic DNA as template using primer pair SEQ ID NO. 47 and SEQ ID NO. 48 (to generate a product of 1526 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 49, SEQ ID NO. 50 and SEQ ID NO. 8.

Rhodococcus jostii SAND007, described above, is transformed with plasmid pSAND008 by electroporation, using standard methods, after which the cell suspension is spread onto LB agar plates with kanamycin. Single colonies are then transferred to LB agar plates containing 10% sucrose. Single colonies are checked for successful disruption of KstD2 by PCR using primer pair SEQ ID NO. 49 and SEQ ID NO. 50, where presence of a 1350 bp PCR product and absence of a 2862 bp PCR product indicates successful disruption. The resulting strain is named Rhodococcus jostii SAND008.

Plasmid pSAND009, to generate a ΔKstD3 (RHA1_RS28305) mutant, is constructed as follows. Plasmid pK18mobsacB is cleaved with restriction enzymes BamHI and PstI. The upstream homology sequence is amplified by PCR using genomic DNA as template using primer pair SEQ ID NO. 51 and SEQ ID NO. 52 (to generate a product of 1529 bp). The downstream homology sequence is amplified by PCR using genomic DNA as template using primer pair SEQ ID NO. 53 and SEQ ID NO. 54 (to generate a product of 1531 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 55, SEQ ID NO. 56 and SEQ ID NO. 8.

Rhodococcus jostii SAND008, described above, is transformed with plasmid pSAND009 by electroporation, using standard methods, after which the cell suspension is spread onto LB agar plates with kanamycin. Single colonies are then transferred to LB agar plates containing 10% sucrose. Single colonies are checked for successful disruption of KstD3 by PCR using primer pair SEQ ID NO. 55 and SEQ ID NO. 56, where presence of a 1361 bp PCR product and absence of a 2927 bp PCR product indicates successful disruption. The resulting strain is named Rhodococcus jostii SAND009.

Plasmid pSAND010, to generate a ΔKstD3b (RHA1_RS28380) mutant, is constructed as follows. Plasmid pK18mobsacB is cleaved with restriction enzymes BamHI and PstI. The upstream homology sequence is amplified by PCR using genomic DNA as template using primer pair SEQ ID NO. 57 and SEQ ID NO. 58 (to generate a product of 1545 bp). The downstream homology sequence is amplified by PCR using genomic DNA as template using primer pair SEQ ID NO. 59 and SEQ ID NO. 60 (to generate a product of 1500 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 61, SEQ ID NO. 62 and SEQ ID NO. 8.

Rhodococcus jostii SAND009, described above, is transformed with plasmid pSAND010 by electroporation, using standard methods, after which the cell suspension is spread onto LB agar plates with kanamycin. Single colonies are then transferred to LB agar plates containing 10% sucrose. Single colonies are checked for successful disruption of KstD3b by PCR using primer pair SEQ ID NO. 61 and SEQ ID NO. 62, where presence of a 1381 bp PCR product and absence of a 2785 bp PCR product indicates successful disruption. The resulting strain is named Rhodococcus jostii SAND010.

Plasmid pSAND011, to generate a ΔKstD4 (RHA1_RS40180) mutant, is constructed as follows. Plasmid pK18mobsacB is cleaved with restriction enzymes BamHI and PstI. The upstream homology sequence is amplified by PCR using genomic DNA as template using primer pair SEQ ID NO. 63 and SEQ ID NO. 64 (to generate a product of 1548 bp). The downstream homology sequence is amplified by PCR using genomic DNA as template using primer pair SEQ ID NO. 65 and SEQ ID NO. 66 (to generate a product of 1542 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 67, SEQ ID NO. 68 and SEQ ID NO. 8.

Rhodococcus jostii SAND010, described above, is transformed with plasmid pSAND011 by electroporation, using standard methods, after which the cell suspension is spread onto LB agar plates with kanamycin. Single colonies are then transferred to LB agar plates containing 10% sucrose. Single colonies are checked for successful disruption of KstD4 by PCR using primer pair SEQ ID NO. 67 and SEQ ID NO. 68, where presence of a 1413 bp PCR product and absence of a 3027 bp PCR product indicates successful disruption. The resulting strain is named Rhodococcus jostii SAND011.

Plasmid pSAND012, to generate a ΔKstD4b (RHA1_RS40260) mutant, is constructed as follows. Plasmid pK18mobsacB is cleaved with restriction enzymes BamHI and PstI. The upstream homology sequence is amplified by PCR using genomic DNA as template using primer pair SEQ ID NO. 69 and SEQ ID NO. 70 (to generate a product of 1590 bp). The downstream homology sequence is amplified by PCR using genomic DNA as template using primer pair SEQ ID NO. 71 and SEQ ID NO. 72 (to generate a product of 1523 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 73, SEQ ID NO. 74 and SEQ ID NO. 8.

Rhodococcus jostii SAND011, described above, is transformed with plasmid pSAND012 by electroporation, using standard methods, after which the cell suspension is spread onto LB agar plates with kanamycin. Single colonies are then transferred to LB agar plates containing 10% sucrose. Single colonies are checked for successful disruption of KstD4b by PCR using primer pair SEQ ID NO. 73 and SEQ ID NO. 74, where presence of a 1451 bp PCR product and absence of a 3002 bp PCR product indicates successful disruption. The resulting strain is named Rhodococcus jostii SAND012.

### Example 3. Fermentation of Rhodococcus jostii SAND001-SAND012 and Isolation of KCDA/KCEA

Strains Rhodococcus jostii SAND001, Rhodococcus jostii SAND002, Rhodococcus jostii SAND003, Rhodococcus jostii SAND004, Rhodococcus jostii SAND005, Rhodococcus jostii SAND006, Rhodococcus jostii SAND007, Rhodococcus jostii SAND008, Rhodococcus jostii SAND009, Rhodococcus jostii SAND010, Rhodococcus jostii SAND011, Rhodococcus jostii SAND012 are independently cultured according to the methods such as those described in U.S. Pat. No. 4,362,815. In one method, 500 mL Erlenmeyer flasks containing 100 mL of sterile culture medium (0.06% Cholesterol or Phytosterol, such as β-Sitosterol, 0.06% Tween 80 (Polyoxyethylene sorbitane mono-oleate), 0.8% Peptone, 0.9% Yeast Extract, 0.3% Glucose, made up with water and adjusted to pH 7.2) are inoculated with the mutant strain. These are incubated for 62 hours at 30° C. with shaking at 150 rpm. The flasks are then made up with sterile BRIJ 35 (polyoxyethylene monolauryl ether) to a final concentration of 0.1% and a further quantity of the sterol feed, such as Cholesterol or Phytosterol to a final quantity of 0.1%. The flasks are then incubated for a further 120 hours at 30° C. with shaking at 150 rpm.

Products, including KCEA and KCDA are extracted from the broth using standard methods, such as those described in Ahmad et al., 1991 and McDonald et al. 2012. In one method, the culture is extracted into an equal volume of ethyl acetate, followed by evaporation, then the sterol of interest is purified using chromatography.

### Example 4. Generation of Knockout Mutants of FadE34 CasC Homologues in Rhodococcus sp. DSM1444 or DSM1445′

Genomic DNA is isolated as described in the general methods and sequenced using Illumina sequencing with hiSeq PCR-free paired-end data and mate pair. Assembly annotation and analysis are performed as described in the general methods.

Homologues of genes encoding FadE34 and CasC are identified by either a protein BLAST search against the assembled, annotated genome or a tblastn search against the obtained nucleotide sequence using SEQ ID NO. 75 and SEQ ID NO. 76 as search queries.

The obtained BLAST hits with the highest bit-scores are targeted for disruption. Each mutant is tested for interruption of cholesterol or phytosterol side-chain degradation according to the methods described in example 6, screening for KCDA and KCEA as well as any alternative degradation products that may form (Ruprecht et al., 2015). If none are present in the broths, BLAST hits with the next highest homology to SEQ ID NO. 75 and SEQ ID NO. 76 are targeted for disruption. Plasmids used to disrupt target genes are constructed as described below.

Plasmid pSAND013, to generate a ΔFadE34 mutant, is constructed as follows. Using the annotated genome sequence as a guide, a primer pair is designed such that they amplify a sequence starting 1-2 kb upstream of the FadE34 homologue start codon and ending a small number of codons, e.g. 10-25 codons, downstream of the FadE34 homologue start codon, with the intention to make an in-frame deletion.

Using the annotated genome sequence as a guide, a primer pair designed such that they amplify a sequence starting a small number of codons, e.g. 10-25 codons, upstream of the FadE34 homologue stop codon and ending 1-2 kb downstream of the FadE34 homologue stop codon, with the intention to make an in-frame deletion.

Further, both sets of primers are designed such that they allow convenient cloning into pK18mobsacB or another suitable vector, either by ligation, InFusion cloning, Gibson assembly or another method of choice. The plasmid is constructed by amplifying the upstream and downstream homology sequences by common PCR methods and subsequent cloning into the vector of choice. Correct assembly of the vector and identity of the inserts are confirmed by restriction digest and sequencing using suitable primers.

Rhodococcus sp. DSM1444 (ATCC31459) or DSM1445 (ATCC31460), microbial strains capable of producing 3-oxo-pregna-1,4-diene-20-carboxyl acid (also known as 1,4-BNC; 3-oxo-23,24-bisnorchola-1,4-dien-22-oic acid), 3-oxo-pregna-4-ene-20-carboxyl acid (also known as 4-BNC; 23,24-bis-nor-cholesta-4-ene-22-oic acid), 4-androstene-3,17-dione (AD) and 1,4-androstadiene-3,17-dione (ADD), is transformed with plasmid pSAND013 by electroporation, using standard methods, after which the cell suspension is spread onto TSA plates with kanamycin or another suitable antibiotic if a plasmid other than pK18mobsacB is used. Single colonies are then transferred to fresh TSA plates and secondary recombinants are selected using suitable methods, e.g. by counter-selection against pK18mobsacB with 10% sucrose. Single colonies are checked for successful disruption of FadE34 by PCR using suitable primer pairs. The resulting strain is named Rhodococcus sp. SAND013.

Plasmid pSAND014, to generate a ΔCasC mutant, is constructed as follows. Using the annotated genome sequence as a guide, a primer pair designed such that they amplify a sequence starting 1-2 kb upstream of the CasC homologue start codon and ending a small number of codons, e.g. 10-25 codons, downstream of the CasC homologue start codon, with the intention to make an in-frame deletion.

Using the annotated genome sequence as a guide, a primer pair designed such that they amplify a sequence starting a small number of codons, e.g. 10-25 codons, upstream of the CasC homologue stop codon and ending 1-2 kb downstream of the CasC homologue stop codon, with the intention to make an in-frame deletion.

Further, both sets of primers are designed such that they allow convenient cloning into pK18mobsacB or another suitable vector, either by ligation, InFusion cloning, Gibson assembly or another method of choice. The plasmid is constructed by amplifying the upstream and downstream homology sequences by common PCR methods and subsequent cloning into the vector of choice. Correct assembly of the vector and identity of the inserts are confirmed by restriction digest and sequencing using suitable primers.

Rhodococcus sp. SAND013 is transformed with plasmid pSAND014 by electroporation, using standard methods, after which the cell suspension is spread onto TSA plates with kanamycin or another suitable antibiotic if a plasmid other than pK18mobsacB is used. Single colonies are then transferred to fresh TSA plates and secondary recombinants are selected using suitable methods, e.g. by counter-selection against pK18mobsacB with 10% sucrose. Single colonies are checked for successful disruption of CasC by PCR using suitable primer pairs. The resulting strain is named Rhodococcus sp. SAND014.

### Example 5. Generation of Knockout Mutants of KstD Homologues in Rhodococcus sp. SAND014

Homologues of genes encoding KstD are identified by either a protein BLAST search against the annotated genome or a tblastn search against the obtained nucleotide sequence using SEQ ID NO. 101 as search query. The following procedure serves as an example and is carried out for each of the homologues to be knocked out.

Plasmid pSAND019, to generate a ΔKstD mutant, is constructed as follows. Using the annotated genome sequence as a guide, a primer pair is designed such that they amplify a sequence starting 1-2 kb upstream of the KstD start codon and ending a small number of codons, e.g. 10-25 codons, downstream of the KstD start codon, with the intention to make an in-frame deletion.

Using the annotated genome sequence as a guide, a primer pair designed such that they amplify a sequence starting a small number of codons, e.g. 10-25 codons, upstream of the KstD stop codon and ending 1-2 kb downstream of the KstD stop codon, with the intention to make an in-frame deletion.

Further, both sets of primers are designed such that they allow convenient cloning into pK18mobsacB or another suitable vector, either by ligation, InFusion cloning, Gibson assembly or another method of choice. The plasmid is constructed by amplifying the upstream and downstream homology sequences by common PCR methods and subsequent cloning into the vector of choice. Correct assembly of the vector and identity of the inserts are confirmed by restriction digest and sequencing using suitable primers.

Rhodococcus sp. SAND014 or a derivative thereof is transformed with plasmid pSAND019 by electroporation, using standard methods, after which the cell suspension is spread onto TSA plates with kanamycin or another suitable antibiotic if a plasmid other than pK18mobsacB is used. Single colonies are then transferred to fresh TSA plates and secondary recombinants are selected using suitable methods, e.g. by counter-selection against pK18mobsacB with 10% sucrose. Single colonies are checked for successful disruption of KstD by PCR using suitable primer pairs, designed using the annotated genome sequence as a guide. The resulting strain is named Rhodococcus sp. SAND015.

### Example 6. Fermentation of Rhodococcus sp. SAND013-SAND015 and Isolation of KCDA/KCEA

Strains Rhodococcus sp. SAND013, Rhodococcus sp. SAND014 and Rhodococcus sp. SAND015 are independently cultured according to standard methods such as those described in U.S. Pat. No. 4,362,815. In one method, 500 mL Erlenmeyer flasks containing 100 mL of sterile culture medium (0.06% Cholesterol or Phytosterol, such as β-Sitosterol, 0.06% Tween 80 (Polyoxyethylene sorbitane mono-oleate), 0.8% Peptone, 0.9% Yeast Extract, 0.3% Glucose, made up with water and adjusted to pH 7.2) are inoculated with the mutant strains. These are incubated for 62 hours at 30° C. with shaking at 150 rpm. The flasks are then made up with sterile BRIJ 35 (Polyoxyethylene monolauryl ether) to a final concentration of 0.1% and a further quantity of the sterol feed, such as Cholesterol or Phytosterol to a final quantity of 0.1%. The flasks are then incubated for a further 120 hours at 30° C. with shaking at 150 rpm.

Products, including KCEA and KCDA are extracted from the broth using standard methods, such as those described in Ahmad et al., 1991 and McDonald et al. 2012. In one method, the culture is extracted with an equal volume of ethyl acetate, followed by evaporation, then the sterol of interest is purified using chromatography.

### Example 7. Generation of Knockout Mutants of FadE34 CasC and KshB in Mycobacterium smegmatis mc(2) 155

Plasmid pSAND015, to generate a ΔFadE34 (MSMEG_6041) mutant, is constructed as follows. Plasmid pK18mobsacB is cleaved with restriction enzymes BamHI and PstI. The upstream homology sequence is amplified by PCR using genomic DNA as template using primer pair SEQ ID NO. 77 and SEQ ID NO. 78 (to generate a product of 1530 bp). The downstream homology sequence is amplified by PCR using genomic DNA as template using primer pair SEQ ID NO. 79 and SEQ ID NO. 80 (to generate a product of 1536 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 81, SEQ ID NO. 82 and SEQ ID NO. 8.

Mycobacterium smegmatis mc(2) 155 (ATCC 700084) is transformed with plasmid pSAND015 by electroporation, using standard methods, after which the cell suspension is spread onto complex 7H9 agar plates with kanamycin. Single colonies are then transferred to complex 7H9 agar plates containing 10% sucrose. Single colonies are checked for successful disruption of FadE34 by PCR using primer pair SEQ ID NO. 81 and SEQ ID NO. 82, where presence of a 1341 bp PCR product and absence of a 3369 bp PCR product indicates successful disruption. The resulting strain is named Mycobacterium smegmatis SAND016.

Plasmid pSAND016, to generate a ΔCasC (MSMEG_0603) mutant, is constructed as follows. Plasmid pK18mobsacB is cleaved with restriction enzymes BamHI and PstI. The upstream homology sequence is amplified by PCR using genomic DNA as template using primer pair SEQ ID NO. 83 and SEQ ID NO. 84 (to generate a product of 1536 bp). The downstream homology sequence is amplified by PCR using genomic DNA as template using primer pair SEQ ID NO. 85 and SEQ ID NO. 86 (to generate a product of 1537 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 87, SEQ ID NO. 88 and SEQ ID NO. 8.

Mycobacterium smegmatis SAND016, described above, is transformed with plasmid pSAND016 by electroporation, using standard methods, after which the cell suspension is spread onto complex 7H9 agar plates with kanamycin. Single colonies are then transferred to complex 7H9 agar plates containing 10% sucrose. Single colonies are checked for successful disruption of CasC by PCR using primer pair SEQ ID NO. 87 and SEQ ID NO. 88, where presence of a 1346 bp PCR product and absence of a 3428 bp PCR product indicates successful disruption. The resulting strain is named Mycobacterium smegmatis SAND017.

Plasmid pSAND017, to generate a ΔKshB (MSMEG_6039) mutant, is constructed as follows. Plasmid pK18mobsacB is cleaved with restriction enzymes BamHI and PstI. The upstream homology sequence is amplified by PCR using genomic DNA as template using primer pair SEQ ID NO. 89 and SEQ ID NO. 90 (to generate a product of 1526 bp). The downstream homology sequence is amplified by PCR using genomic DNA as template using primer pair SEQ ID NO. 91 and SEQ ID NO. 92 (to generate a product of 1539 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 93, SEQ ID NO. 94 and SEQ ID NO. 8.

Mycobacterium smegmatis SAND017, described above, is transformed with plasmid pSAND017 by electroporation, using standard methods, after which the cell suspension is spread onto complex 7H9 agar plates with kanamycin. Single colonies are then transferred to complex 7H9 agar plates containing 10% sucrose. Single colonies are checked for successful disruption of KshB by PCR using primer pair SEQ ID NO. 93 and SEQ ID NO. 94, where presence of a 1395 bp PCR product and absence of a 2268 bp PCR product indicates successful disruption. The resulting strain is named Mycobacterium smegmatis SAND018.

### Example 8. Generation of a ΔKstD Knockout Mutant in Mycobacterium smegmatis SAND018

Plasmid pSAND018, to generate a ΔKstD (MSMEG_5941) mutant, is constructed as follows. Plasmid pK18mobsacB is cleaved with restriction enzymes BamHI and PstI. The upstream homology sequence is amplified by PCR using genomic DNA as template using primer pair SEQ ID NO. 95 and SEQ ID NO. 96 (to generate a product of 1522 bp). The downstream homology sequence is amplified by PCR using genomic DNA as template using primer pair SEQ ID NO. 97 and SEQ ID NO. 98 (to generate a product of 1527 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 99, SEQ ID NO. 100 and SEQ ID NO. 8.

Mycobacterium smegmatis SAND018 is transformed with plasmid pSAND018 by electroporation, using standard methods, after which the cell suspension is spread onto complex 7H9 agar plates with kanamycin. Single colonies are then transferred to complex 7H9 agar plates containing 10% sucrose. Single colonies are checked for successful disruption of KstD by PCR using primer pair SEQ ID NO. 99 and SEQ ID NO. 100, where presence of a 1354 bp PCR product and absence of a 2929 bp PCR product indicates successful disruption. The resulting strain is named Mycobacterium smegmatis SAND019.

### Example 9. Fermentation of Mycobacterium smegmatis SAND016-SAND019 and Isolation of KCDA/KCEA

Strains Mycobacterium smegmatis SAND016, Mycobacterium smegmatis SAND017, Mycobacterium smegmatis SAND018 and Mycobacterium smegmatis SAND019 are independently cultured according to the methods such as those described in (Galán et al., 2017).

In one method, 500 mL Erlenmeyer flasks containing 100 mL of sterile Middlebrook 7H9 broth medium without albumin-dextrose-catalase supplement containing 9 mM glycerol and 0.4 g/L cholesterol or phytosterol, such as β-sitosterol are inoculated with the mutant strain. These are incubated for 100 hours at 37° C. with shaking at 200 rpm.

Products, including KCEA and KCDA are extracted from the broth using standard methods, such as those described in Ahmad et al., 1991 and McDonald et al. 2012. In one method, the culture is extracted with an equal volume of ethyl acetate, followed by evaporation, then the sterol of interest is purified using chromatography.

### Example 10. Generation of ΔChsE3 and ΔCasC Knockout Mutants in Mycobacterium neoaurum NRRL B-3805

Plasmid pSAND019, to generate a ΔChsE3 (GENE ID MyAD_RS24250) mutant, is constructed as follows. Plasmid pK18mobsacB is cleaved with restriction enzymes BamHI and HindIII. The upstream homology sequence is amplified by PCR using genomic DNA as template using primer pair SEQ ID NO. 121 and SEQ ID NO. 102 (to generate a product of 2034 bp). The downstream homology sequence is amplified by PCR using genomic DNA as template using primer pair SEQ ID NO. 103 and SEQ ID NO. 104 (to generate a product of 2034 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 105, SEQ ID NO. 106, SEQ ID NO. 107 and SEQ ID NO. 8.

Mycobacterium neoaurum NRRL B-3805 is transformed with plasmid pSAND019 by electroporation, using standard methods, after which the cell suspension is spread onto M3 agar plates with kanamycin. Single colonies are then transferred to M3 agar plates containing 5% sucrose. Single colonies are checked for successful disruption of ChsE3 by PCR using primer pair SEQ ID NO. 105 and SEQ ID NO. 107, where presence of a 2352 bp PCR product and absence of a 4434 bp PCR product indicates successful disruption. The resulting strain is named Mycobacterium neoaurum SAND020.

Plasmid pSAND020, to generate a ΔCasC (GENE ID MyAD_RS03655) mutant, is constructed as follows. Plasmid pK18mobsacB is cleaved with restriction enzymes BamHI and HindIII. The upstream homology sequence is amplified by PCR using genomic DNA as template using primer pair SEQ ID NO. 108 and SEQ ID NO. 109 (to generate a product of 1996 bp). The downstream homology sequence is amplified by PCR using genomic DNA as template using primer pair SEQ ID NO. 110 and SEQ ID NO. 111 (to generate a product of 2004 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 112, SEQ ID NO. 113, SEQ ID NO. 114 and SEQ ID NO. 8.

Mycobacterium neoaurum SAND020 is transformed with plasmid pSAND020 by electroporation, using standard methods, after which the cell suspension is spread onto M3 agar plates with kanamycin. Single colonies are then transferred to M3 agar plates containing 5% sucrose. Single colonies are checked for successful disruption of CasC by PCR using primer pair SEQ ID NO. 112 and SEQ ID NO. 114, where presence of a 2281 bp PCR product and absence of a 4399 bp PCR product indicates successful disruption. The resulting strain is named Mycobacterium neoaurum SAND021.

### Example 11: Generation of Knockout Mutants of KstD Homologues in Mycobacterium neoaurum SAND020-SAND021

Plasmid pSAND021, to generate a ΔKstD (GENE ID MyAD_RS23810) mutant, is constructed as follows. Plasmid pK18mobsacB is cleaved with restriction enzymes BamHI and HindIII. The upstream homology sequence is amplified by PCR using genomic DNA as template using primer pair SEQ ID NO. 115 and SEQ ID NO. 116 (to generate a product of 2037 bp). The downstream homology sequence is amplified by PCR using genomic DNA as template using primer pair SEQ ID NO. 117 and SEQ ID NO. 118 (to generate a product of 2022 bp). Both PCR products are inserted into cleaved pK18mobsacB using the InFusion Cloning kit (Takara Bio.) following the manufacturer's instructions. Insertion and identity of the inserts are confirmed by restriction digest and sequencing using primers SEQ ID NO. 5, SEQ ID NO. 119, SEQ ID NO. 120, SEQ ID NO. 121 and SEQ ID NO. 8.

Mycobacterium neoaurum SAND020 is transformed with plasmid pSAND021 by electroporation, using standard methods, after which the cell suspension is spread onto M3 agar plates with kanamycin. Single colonies are then transferred to M3 agar plates containing 5% sucrose. Single colonies are checked for successful disruption of KstD by PCR using primer pair SEQ ID NO. 119 and SEQ ID NO. 121, where presence of a 2572 bp PCR product and absence of a 4081 bp PCR product indicates successful disruption. The resulting strain is named Mycobacterium neoaurum SAND022.

Mycobacterium neoaurum SAND021 is transformed with plasmid pSAND021 by electroporation, using standard methods, after which the cell suspension is spread onto M3 agar plates with kanamycin. Single colonies are then transferred to M3 agar plates containing 5% sucrose. Single colonies are checked for successful disruption of KstD by PCR using primer pair SEQ ID NO. 119 and SEQ ID NO. 121, where presence of a 2572 bp PCR product and absence of a 4081 bp PCR product indicates successful disruption. The resulting strain is named Mycobacterium neoaurum SAND023.

### Example 12: Fermentation of Mycobacterium neoaurum SAND020-SAND023 and Isolation of KCDA/KCEA

Strains Mycobacterium neoaurum SAND020, Mycobacterium neoaurum SAND021, Mycobacterium neoaurum SAND022 and Mycobacterium neoaurum SAND023, are independently cultured according to the methods such as those described in the literature (e.g. Marsheck et al., 1972). In one method, one-liter Erlenmeyer flasks containing 250 ml of medium A were inoculated with a 48-hr liquid culture of the strain and incubated at 31° C. with shaking at 250 RPM (2.5 cm throw). After 48 hours, 100 mg of powdered sterol (such as Cholesterol or Phytosterol, such as β-Sitosterol) is added to each flask and the incubation was continued for 72 to 96 hours.

Products, including KCEA and KCDA are extracted from the broth using standard methods, such as those described in Ahmad et al., 1991 and McDonald et al. 2012. In one method, the culture is extracted into an equal volume of ethyl acetate, followed by evaporation, then the sterol of interest is purified using chromatography.

### Example 13: Generation of a Knockout Mutant of the FadE34 Homologue in Rhodococcus sp. DSM1444

Plasmid pSAND022, used as a backbone for gene knockout plasmids, was constructed as follows. Plasmid pKC1132 (Bierman et al., 1992) was linearised with restriction enzyme PciI. A sequence encoding Bacillus subtilis gene sacB (SEQ ID NO. 122) was digested with restriction enzyme NcoI and inserted into cleaved pKC1132 by ligation following standard methods. Insertion and identity of the insert was confirmed by restriction digest. The resulting plasmid was labelled pSAND022.

Plasmid pSAND023, to generate a ΔFadE34 homologue mutant, was constructed as follows. Plasmid pSAND022 was cleaved with restriction enzymes BglII and HindIII. The upstream homology sequence was amplified by PCR using genomic DNA as template using primer pair SEQ ID NO. 123 and SEQ ID NO. 124 (to generate a product of 1549 bp). This PCR product was digested with restriction enzymes BamHI and SpeI. The downstream homology sequence was amplified by PCR using genomic DNA as template using primer pair SEQ ID NO. 125 and SEQ ID NO. 126 (to generate a product of 1579 bp.). This PCR product was digested with restriction enzymes HindIII and SpeI. Both digested PCR products were inserted into cleaved pSAND022 by ligation following standard methods. Insertion and identity of the inserts were confirmed by restriction digest and sequencing using primers SEQ ID NO. 5, SEQ ID NO. 127, SEQ ID NO. 128 and SEQ ID NO. 8.

Rhodococcus sp. DSM1444 was transformed with plasmid pSAND023 by electroporation, using standard methods, after which the cell suspension was spread onto TSA agar plates with apramycin. Single colonies were grown up in 50 ml TSB in 250-ml Erlenmeyer flasks at 30° C. shaking at 250 RPM for 2 days. Dilutions of this culture were plated onto TSA agar plates containing 10% sucrose. Single colonies were checked for successful disruption of the FadE34 homologue by PCR using primer pair SEQ ID NO. 129 and SEQ ID NO. 130, where presence of a 3213 bp PCR product and absence of a 5223 bp PCR product indicated successful disruption. The identity of the 3213 bp PCR product was confirmed by sequencing with primers SEQ ID NO. 127 and SEQ ID NO. 128. The resulting strain was named Rhodococcus neoaurum SAND024.

### Example 14: Fermentation of Rhodococcus sp. SAND024 and Analysis of Culture Extracts

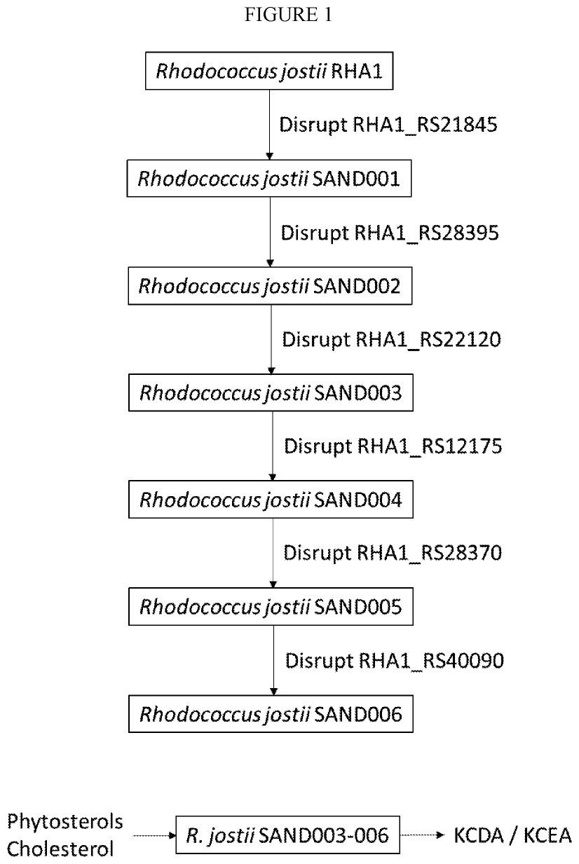

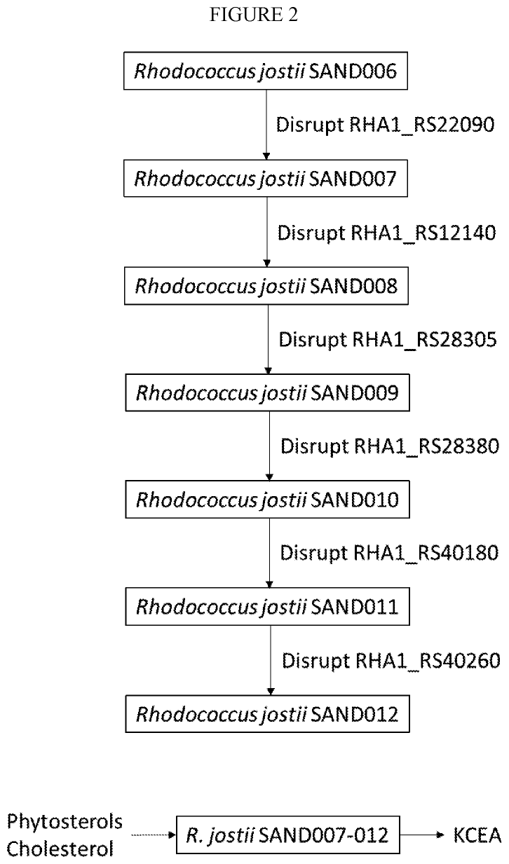

5 mL sterile culture medium (0.8% Peptone, 0.9% Yeast Extract, 0.3% Glucose, 0.06% Tween 80, made up with water and adjusted to pH 7.2 prior to sterilization) in a 50-mL Falcon tube was inoculated with Rhodococcus sp. SAND024 and incubated at 30° C. with shaking at 250 RPM for 24 hours, to be used as the seed culture. 40 mL sterile culture medium (0.8% Peptone, 0.9% Yeast Extract, 0.3% Glucose, 0.06% Tween 80, made up with water and adjusted to pH 7.2 prior to sterilization) containing 0.06% Cholesterol in a 250-mL conical shake flask was inoculated with 0.709 mL of the seed culture and incubated at 30° C. with shaking at 250 RPM for 2 days. 40 mg Cholesterol was dissolved in 2 mL ethanol, 0.13 mL BRIJ-35 (30% w/v solution in water) was added to this solution and the suspension was added to the culture. The culture was incubated at 30° C. with shaking at 250 RPM for 5 days. 0.8 mL broth was withdrawn from the culture and extracted with 0.8 mL of ethyl acetate by shaking for 1 hour. Phases were separated by centrifugation, and 0.3 mL of the solvent phase was transferred to a clean tube and evaporated. The pellet was dissolved in 0.15 mL of methanol and analyzed by HPLC-MS (see General methods). Production of KCEA and KCDA was confirmed in comparison to an isolated standard (see FIGS. 1 and 2).

### Example 15: Generation of a Knockout Mutant of KshA1 in Rhodococcus jostii RHA1

Plasmid pSAND024, to generate a ΔKshA1 mutant, was constructed as follows. Plasmid pUC19 was cleaved with restriction enzymes EcoRI and HindIII. The upstream homology sequence was amplified by PCR using genomic DNA as template using primer pair SEQ ID NO. 131 and SEQ ID NO. 132 (to generate a product of 1544 bp). This PCR product was digested with restriction enzymes EcoRI and SpeI. The downstream homology sequence was amplified by PCR using genomic DNA as template using primer pair SEQ ID NO. 133 and SEQ ID NO. 134 (to generate a product of 1578 bp.). This PCR product was digested with restriction enzymes HindIII and SpeI. Both digested PCR products were inserted into cleaved pUC19 by ligation following standard methods. Insertion and identity of the inserts were confirmed by restriction digest and sequencing using primers SEQ ID NO. 5, SEQ ID NO. 19 and SEQ ID NO. 8. The resulting plasmid was subsequently digested with restriction enzymes EcoRI and HindIII. Fragments were separated by agarose gel electrophoresis and a fragment of 2941 bp, containing the upstream homology region and the downstream homology region, was isolated using the Macherey-Nagel Nucleospin Gel and PCR clean-up Kit, following the manufacturer's instructions. Plasmid pSAND022 was digested with restriction enzymes EcoRI and HindIII. Fragments were separated by agarose gel electrophoresis and a fragment of 5429 bp was isolated using the Macherey-Nagel Nucleospin Gel and PCR Clean-up Kit, following the manufacturer's instructions. Digested pSAND022 was ligated with the 2941 bp DNA fragment containing the upstream homology region and the downstream homology region following standard methods.

Rhodococcus jostii RHA1 was transformed with plasmid pSAND024 by electroporation, using standard methods, after which the cell suspension was spread onto LB agar plates with apramycin. Single colonies were grown up in 3 ml LB in 50-ml Falcon tubes at 28° C. shaking at 250 RPM for 24 hours. Dilutions of this culture were plated onto LB agar plates containing 10% sucrose. Single colonies were checked for successful disruption of KshA1 by PCR using primer pair SEQ ID NO. 135 and SEQ ID NO. 136, where presence of a 3147 bp PCR product and absence of a 4209 bp PCR product indicated successful disruption. The identity of the 3147 bp PCR product was confirmed by sequencing with primers SEQ ID NO. 19 and SEQ ID NO. 20. The resulting strain was named Rhodococcus sp. SAND025.

### Example 16: Generation of a Knockout Mutant of FadE34 in Rhodococcus sp. SAND025

Plasmid pSAND025, to generate a ΔFadE34 mutant, was constructed as follows. Plasmid pSAND022 was cleaved with restriction enzymes EcoRI and HindIII. The upstream homology sequence was amplified by PCR using genomic DNA as template using primer pair SEQ ID NO. 1 and SEQ ID NO. 137 (to generate a product of 1535 bp). This PCR product was digested with restriction enzymes EcoRI and SpeI. The downstream homology sequence was amplified by PCR using genomic DNA as template using primer pair SEQ ID NO. 138 and SEQ ID NO. 4 (to generate a product of 1540 bp.). This PCR product was digested with restriction enzymes HindIII and SpeI. Both digested PCR products were inserted into cleaved pSAND022 by ligation following standard methods. Insertion and identity of the inserts were confirmed by restriction digest and sequencing using primers SEQ ID NO. 5, SEQ ID NO. 6, SEQ ID NO. 7 and SEQ ID NO. 8.

Rhodococcus sp. SAND025 was transformed with plasmid pSAND025 by electroporation, using standard methods, after which the cell suspension was spread onto LB agar plates with apramycin. Single colonies were grown up in 3 ml LB in 50-ml Falcon tubes at 28° C. shaking at 250 RPM for 24 hours. Dilutions of this culture were plated onto LB agar plates containing 10% sucrose. Single colonies were checked for successful disruption of FadE34 by PCR using primer pair SEQ ID NO. 139 and SEQ ID NO. 140, where presence of a 3203 bp PCR product and absence of a 5255 bp PCR product indicated successful disruption. The identity of the 3203 bp PCR product was confirmed by sequencing with primers SEQ ID NO. 6 and SEQ ID NO. 7. The resulting strain was named Rhodococcus sp. SAND026.

### Example 17: Generation of a Knockout Mutant of CasC in Rhodococcus sp. SAND026

Plasmid pSAND026, to generate a ΔCasC mutant, was constructed as follows. Plasmid pSAND022 was cleaved with restriction enzymes BamHI and HindIII. The upstream homology sequence was amplified by PCR using genomic DNA as template using primer pair SEQ ID NO. 9 and SEQ ID NO. 141 (to generate a product of 1543 bp). This PCR product was digested with restriction enzymes BglII and SpeI. The downstream homology sequence was amplified by PCR using genomic DNA as template using primer pair SEQ ID NO. 12 and SEQ ID NO. 142 (to generate a product of 1541 bp.). This PCR product was digested with restriction enzymes HindIII and SpeI. Both digested PCR products were inserted into cleaved pSAND022 by ligation following standard methods. Insertion and identity of the inserts were confirmed by restriction digest and sequencing using primers SEQ ID NO. 5, SEQ ID NO. 13, SEQ ID NO. 14 and SEQ ID NO. 8.

Rhodococcus sp. SAND026 was transformed with plasmid pSAND026 by electroporation, using standard methods, after which the cell suspension was spread onto LB agar plates with apramycin. Single colonies were grown up in 3 ml LB in 50-ml Falcon tubes at 28° C. shaking at 250 RPM for 24 hours. Dilutions of this culture were plated onto LB agar plates containing 10% sucrose. Single colonies were checked for successful disruption of CasC by PCR using primer pair SEQ ID NO. 143 and SEQ ID NO. 144, where presence of a 3066 bp PCR product and absence of a 4506 bp PCR product indicated successful disruption. The identity of the 3066 bp PCR product was confirmed by sequencing with primers SEQ ID NO. 13. The resulting strain was named Rhodococcus sp. SAND027.

### Example 18: Fermentation of Rhodococcus sp. SAND027 and Analysis of Culture Extracts

5 mL sterile culture medium (0.8% Peptone, 0.9% Yeast Extract, 0.3% Glucose, 0.06% Tween 80, made up with water and adjusted to pH 7.2 prior to sterilization) containing 50 μg/mL cholesterol in a 50-mL Falcon tube was inoculated with Rhodococcus sp. SAND027 and incubated at 28° C. with shaking at 250 RPM for 48 hours, to be used as the seed culture. 7 mL sterile culture medium (0.8% Peptone, 0.9% Yeast Extract, 0.3% Glucose, 0.06% Tween 80, made up with water and adjusted to pH 7.2 prior to sterilization) containing 0.06% Cholesterol in a 50-mL Falcon tube was inoculated with seed culture to an optical density at 600 nm of 0.05 and incubated at 28° C. with shaking at 250 RPM for 48 hours. 7 mg Cholesterol was suspended in a small volume of sterile culture medium, mixed with 0.23 μL BRIJ-35 (30% w/v solution in water) and added to the culture. The culture was incubated at 28° C. with shaking at 250 RPM for 5 days. 0.8 mL broth was withdrawn from the culture and extracted with 0.8 mL of ethyl acetate by shaking for 1 hour. Phases were separated by centrifugation, and 0.3 mL of the solvent phase was transferred to a clean tube and evaporated. The pellet was dissolved in 0.15 mL of methanol and analyzed by HPLC-MS (see General Methods). Production of KCEA and KCDA was confirmed in comparison to an isolated standard.

### Example 19: Generation of a ChsE3 Knockout Mutant in Mycobacterium neoaurum NRRL B-3805

Plasmid pSAND027, to generate a ΔChsE3 (MyAD_RS24250) mutant, was constructed as follows. Plasmid pSAND022 was cleaved with restriction enzymes BamHI and HindIII. The upstream homology sequence was amplified by PCR using genomic DNA as template using primer pair SEQ ID NO. 145 and SEQ ID NO. 146 (to generate a product of 2034 bp). The downstream homology sequence was amplified by PCR using genomic DNA as template using primer pair SEQ ID NO. 147 and SEQ ID NO. 148 (to generate a product of 2034 bp). Both PCR products were inserted into cleaved pSAND022 using the InFusion Cloning kit (Takara Bio.) following the manufacturer's instructions. Insertion and identity of the inserts were confirmed by restriction digest and sequencing using primers SEQ ID NO. 5, SEQ ID NO. 105, SEQ ID NO. 106, SEQ ID NO. 107 and SEQ ID NO. 8.

Mycobacterium neoaurum NRRL B-3805 was transformed with plasmid pSAND027 by electroporation, using standard methods, after which the cell suspension was spread onto M3 agar plates with apramycin. Single colonies were transferred to a fresh M3 agar plate with apramycin. Cells from the patches were grown up in 5 mL M3-Tw medium in 50-mL Falcon tubes at 30° C. shaking at 220 RPM for 48 hours. Small volumes of this culture were plated onto M3 agar plates with 5% sucrose. Single colonies were checked for successful disruption of ChsE3 by PCR using primer pair SEQ ID NO. 149 and SEQ ID NO. 150, where absence of a 6.2 kb band and presence of a 4.1 kb band indicates successful disruption. The resulting strain is named Mycobacterium neoaurum SAND028.

### Example 20: Generation of a CasC Knockout Mutant in Mycobacterium neoaurum SAND028

Plasmid pSAND028, to generate a ΔCasC (MyAD_RS03655) mutant, was constructed as follows. Plasmid pSAND022 was cleaved with restriction enzymes BamHI and HindIII. The upstream homology sequence was amplified by PCR using genomic DNA as template using primer pair SEQ ID NO. 151 and SEQ ID NO. 152 (to generate a product of 1996 bp). The downstream homology sequence was amplified by PCR using genomic DNA as template using primer pair SEQ ID NO. 153 and SEQ ID NO. 154 (to generate a product of 2007 bp). Both PCR products were inserted into cleaved pSAND022 using the InFusion Cloning kit (Takara Bio.) following the manufacturer's instructions. Insertion and identity of the inserts were confirmed by restriction digest and sequencing using primers SEQ ID NO. 5, SEQ ID NO. 112, SEQ ID NO. 113, SEQ ID NO. 114 and SEQ ID NO. 8

Mycobacterium neoaurum SAND028 was transformed with plasmid pSAND028 by electroporation, using standard methods, after which the cell suspension was spread onto M3 agar plates with apramycin. Single colonies were transferred to a fresh M3 agar plate with apramycin. Cells from the patches were grown up in 3 mL M3-Tw medium in 50-mL Falcon tubes at 30° C. shaking at 220 RPM for 48 hours. 20 μL of this culture was plated onto M3 agar plates with 5% sucrose. Single colonies were checked for successful disruption of CasC by PCR using primer pair SEQ ID NO. 155 and SEQ ID NO. 156, where absence of a 6.2 kb band and presence of a 4.1 kb band indicates successful disruption. The resulting strain was named Mycobacterium neoaurum SAND029.

### Example 21. Bioconversion of Cholesterol and Beta-Sitosterol into KCEA by Mycobacterium neoaurum SAND029

5 mL M3-Tw medium in a 50-mL Falcon tube was inoculated with Mycobacterium neoaurum SAND029 and incubated at 30° C. with shaking at 220 RPM for 3 days, to be used as the seed culture. After 3 days, cells were harvested from the seed culture by centrifugation (2000×g, 5 minutes) and suspended in 5 mL Mycobacterium minimal bioconversion medium, to be used as the inoculum. 2 flasks containing 40 mL Mycobacterium minimal bioconversion medium were inoculated with 1 mL of the inoculum, to be used as the bioconversion cultures.

Sterol formulations were prepared as follows. 2.1 g hydroxypropyl-beta-cyclodextrin was weighed out and dissolved in 2.1 mL dH₂O with the aid of a sonicator bath, vortex mixer and incubating in an oven set to 60° C. for up to 30 minutes. 45 mg cholesterol and plant sterol mixture (75% beta-sitosterol, 10% campesterol) (Acros Organics) were weighed out and dissolved in 1.93 mL hydroxypropyl-beta-cyclodextrin solution (Roquette) each with the aid of a sonicator bath, vortex mixer and incubating in an oven set to 60° C. for up to 30 minutes.

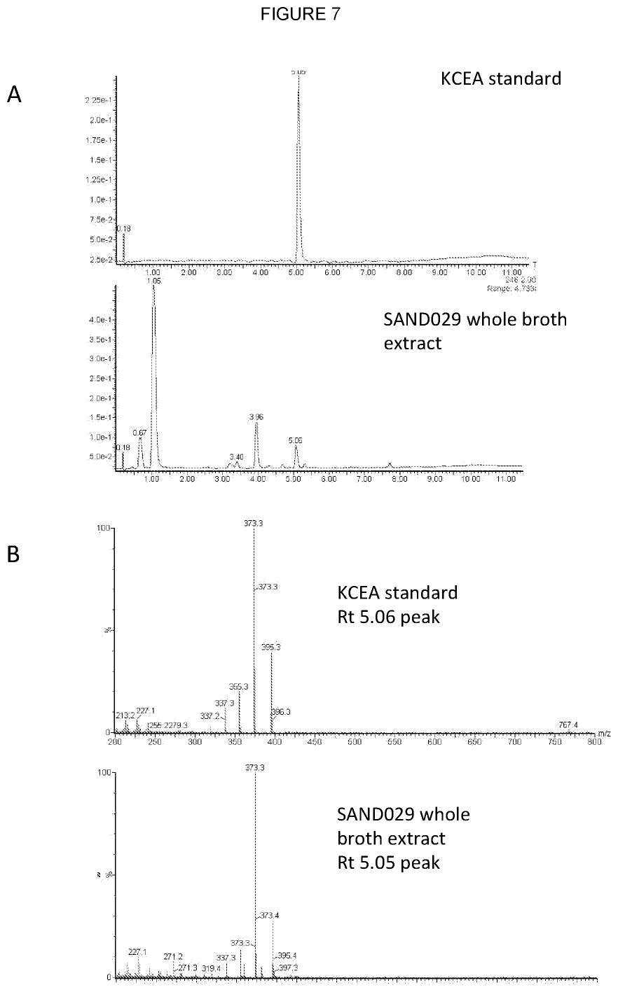

1.72 mL of the cholesterol formulation was added to one of the bioconversion cultures and 1.72 mL of the beta-sitosterol/campesterol formulation was added to the other bioconversion culture. Both bioconversion cultures were incubated at 30° C. with shaking at 220 RPM for 2 or 3 days. 0.8 mL samples were taken at regular intervals and extracted as follows. 15 μL formic acid was mixed into a sample, after which it was extracted with an equal volume ethyl acetate. The solvent layer was evaporated under reduced pressure and the remaining pellet was dissolved in half the original volume of methanol. The methanol solution was analyzed by LCMS (see General Methods) and peaks with an identical retention time and mass spectra profile as seen with a KCEA standard run alongside were seen (see FIG. 7). In addition, peaks with an identical retention time and mass spectra profile as seen with a KCEA-alcohol standard were seen.

### Example 22. Generation of a ChsE1/2 knockout mutant in Mycobacterium neoaurum SAND029

Plasmid pSAND029, to generate a ΔChsE1-ChsE2 (MyAD_RS24025 and MyAD_RS24020) double mutant, was constructed as follows. Plasmid pSAND022 was cleaved with the restriction enzyme NotI. The upstream homology sequence was amplified by PCR using genomic DNA as template using primer pair SEQ ID NO. 157 and SEQ ID NO. 158 (to generate a product of 1831 bp). The downstream homology sequence was amplified by PCR using genomic DNA as template using primer pair SEQ ID NO. 159 and SEQ ID NO. 160 (to generate a product of 1974 bp). Both PCR products were inserted into cleaved pSAND022 using the SLiCE Cloning Method (Zhang et al., 2014) by mixing all fragments and incubation at 37° C. for 15 minutes. Insertion and identity of the inserts were confirmed by restriction digest. The inserts were subsequently sequenced using primers SEQ ID NO. 5, SEQ ID NO. 161, SEQ ID NO. 162, SEQ ID NO. 163 and SEQ ID NO. 8.

Mycobacterium neoaurum SAND029 was transformed with plasmid pSAND029 by electroporation, using standard methods, after which the cell suspension was spread onto M3 agar plates with apramycin. Single colonies were transferred to a fresh M3 agar plate with apramycin. Cells from the patches were grown up in 3 mL M3-Tw medium in 50-mL Falcon tubes at 30° C. shaking at 220 RPM for 48 hours. 20 μL of this culture was plated onto M3 agar plates with 5% sucrose. Single colonies were checked for successful disruption of ChsE3 by PCR using primer pair SEQ ID NO. 164 and SEQ ID NO. 165, where absence of a 6.0 kb band and presence of a 3.9 kb band indicates successful disruption. Integrity of the mutated sequence was confirmed by sequencing of the 3.9 kb PCR product with sequencing primers SEQ ID NO. 160, SEQ ID NO. 161 and SEQ ID NO. 162 The resulting strain was named Mycobacterium neoaurum SAND030.

### Example 23. Bioconversion of Cholesterol into KCEA by Mycobacterium neoaurum SAND030

5 mL M3-Tw medium in a 50-mL Falcon tube was inoculated with Mycobacterium neoaurum SAND030 and incubated at 30° C. with shaking at 220 RPM for 3 days, to be used as the seed culture. After 3 days, cells from 3.5 mL of this seed culture were harvested by centrifugation (2000×g, 5 minutes) and suspended in 3.5 mL Mycobacterium minimal bioconversion medium, to be used as the inoculum. 3 flasks containing 40 mL Mycobacterium minimal bioconversion medium were inoculated with 1 mL of the inoculum, to be used as the bioconversion cultures. Sterol formulations were prepared, for each bioconversion culture, as follows. 2.1 g hydroxypropyl-beta-cyclodextrin was weighed out and dissolved in 2.1 mL dH₂O with the aid of a sonicator bath, vortex mixer and incubating in an oven set to 60° C. for up to 30 minutes. 45 mg cholesterol was weighed out and dissolved in 1.93 mL hydroxypropyl-beta-cyclodextrin solution with the aid of a sonicator bath, vortex mixer and incubating in an oven set to 60° C. for up to 30 minutes.

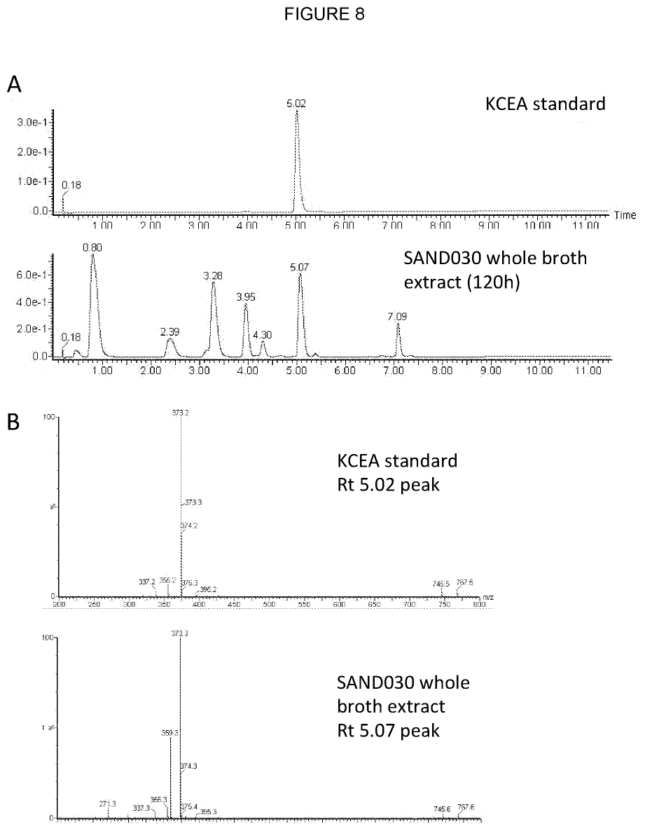

1.72 mL of the cholesterol formulation was added to each of the bioconversion cultures. Both bioconversion cultures were incubated at 30° C. with shaking at 220 RPM for 7 days. 0.5 mL samples were taken at regular intervals and extracted as follows. 10 μL formic acid was mixed into a sample, after which it was extracted with an equal volume ethyl acetate. The solvent layer was evaporated under reduced pressure and the remaining pellet was dissolved in half the original volume of methanol. The methanol solution was analyzed by HPLC-MS (see General Methods) and peaks with an identical retention time and mass spectra profile as seen with a KCEA standard run alongside were seen (see FIG. 8). In addition, peaks with an identical retention time and mass spectra profile as seen with a KCEA-alcohol standard were seen.

To compare the level and efficiency of conversion to KCEA over time between the different strains, levels of KCEA and AD (androstene-3,17-dione) were measured over the course of the study. Table 1 shows the analysis of the maximum concentration and ratio of KCEA to AD maximum concentration. This analysis revealed that Mycobacterium neoaurum SAND030 was more efficient at converting cholesterol to KCEA, as evidenced by a higher maximal measured concentration and ratio of KCEA to AD than Mycobacterium neoaurum SAND029 which was more efficient than Mycobacterium neoaurum NRRL B-3805. Comparison of KCEA concentrations versus the total of all other major catabolic products (AD, BA (bisnoralcohol), KCEA-alcohol) gave similar trends.

TABLE 1
Comparison of maximal KCEA, AD or total AD,
BA and KCEA-alcohol at the same timepoint

| | | | | Total other | Ratio of |
|---|---|---|---|---|---|
| | Maximum | AD | Ratio of | catabolic products | KCEA:other |
| | KCEA (mg/L) | (mg/L) | KCEA:AD | (mg/L) | products |
| Mycobacterium | 30.9 | 470.5 | 0.07 | 601.1 | 0.05 |
| neoaurum | | | | | |
| NRRL B-3805 | | | | | |
| Mycobacterium | 158.2 | 377.9 | 0.42 | 479.6 | 0.33 |
| neoaurum | | | | | |
| SAND029 | | | | | |
| Mycobacterium | 164.7 | 223.2 | 0.74 | 318.0 | 0.52 |
| neoaurum | | | | | |
| SAND030 | | | | | |

### REFERENCES CITED

1. Bibb, L. A., and Hatfull, G. F. (2002). Integration and excision of the Mycobacterium tuberculosis prophage-like element, φRv1: Integration and excision of φRv1. Molecular Microbiology 45, 1515-1526.
2. Galán, B., Uhia, I., Garcia-Fernández, E., Martinez, I., Bahillo, E., de la Fuente, J. L., Barredo, J. L., Fernández-Cabezón, L., and Garcia, J. L. (2017). Mycobacterium smegmatis is a suitable cell factory for the production of steroidic synthons. Microbial Biotechnology 10, 138-150.
3. Geize, R. van der, Hessels, G. I., Gerwen, R. van, Meijden, P. van der, and Dijkhuizen, L. (2001). Unmarked gene deletion mutagenesis of kstD, encoding 3-ketosteroid Δ1-dehydrogenase, in Rhodococcus erythropolis SQ1 using sacB as counter-selectable marker. FEMS Microbiology Letters 205, 197-202.
4. van der Geize, R., Hessels, G. I., van Gerwen, R., Vrijbloed, J. W., van der Meijden, P., and Dijkhuizen, L. (2000). Targeted Disruption of the kstD Gene Encoding a 3-Ketosteroid D1-Dehydrogenase Isoenzyme of Rhodococcus erythropolis Strain SQ1. Appl. Environ. Microbiol. 66, 2029-2036.
5. Green, M. R., and Sambrook, J. (2012). Molecular Cloning: A Laboratory Manual, Fourth Edition (Cold Spring Harbor, N. Y: Cold Spring Harbor Press).
6. Lorraine, J. K. and Smith, M. C. M. Methods Mol Biol. 2017; 1645:93-108. doi: 10.1007/978-1-4939-7183-1_7.
7. Pelicic, V., Reyrat, J. M., and Gicquel, B. (1996). Expression of the Bacillus subtilis sacB gene confers sucrose sensitivity on mycobacteria. Journal of Bacteriology 178, 1197-1199.
8. Pospiech, A., and Neumann, B. (1995). A versatile quick-prep of genomic DNA from Gram-positive bacteria. Trends in Genetics 11, 217-218.
9. Ruprecht, A., Maddox, J., Stirling, A. J., Visaggio, N., and Seah, S. Y. K. (2015). Characterization of Novel Acyl Coenzyme A Dehydrogenases Involved in Bacterial Steroid Degradation. Journal of Bacteriology 197, 1360-1367.
10. Zhang, Y., Werling, U., Ederlmann, W. (2014). Seamless Ligation Cloning Extract (SLiCE) Cloning Method. Methods in Molecular Biology 1116, 235-244.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1) A non-naturally occurring steroid degrading microbial organism comprising the following enzymatic activities disrupted: aerobic 9,10-seco degradation activity and 4-(steroid-17-yl)pentanoyl side chain degradation activity.

2) A non-naturally occurring steroid degrading microbial organism comprising the following enzymatic activities disrupted: steroid 9-alpha-hydroxylation activity and 4-(steroid-17-yl)pentanoyl side chain degradation activity.

3) The non-naturally occurring organism of claim 1 or 2 wherein the 4-(steroid-17-yl)pentanoyl side chain degradation activity comprises activity for 4-(steroid-17-yl)pentanoyl dehydrogenation at the 22,23-position.

4) A non-naturally occurring steroid degrading microbial organism characterized by the following isoenzymes disrupted:
a) one or more 3-ketosteroid-9-alpha-hydroxylase isoenzymes disrupted at an oxygenase subunit, a ferredoxin reductase subunit, or a combination thereof; and
b) one or more acyl-CoA dehydrogenase isoenzymes that act on steroid CoA esters having five carbon side chains.

5) A non-naturally occurring steroid degrading microbial organism characterized by the following enzymatic activities disrupted:
a) one or more 3-ketosteroid-9-alpha-hydroxylase isoenzymes disrupted at an oxygenase subunit, a ferredoxin reductase subunit, or a combination thereof;
b) one or more acyl-CoA dehydrogenase isoenzymes that act on steroid CoA esters having five carbon side chains; and
c) one or more 3-ketosteroid delta-1 dehydrogenase isoenzymes.

6) The non-naturally occurring microorganism of claim 4 or 5 wherein the one or more acyl-CoA dehydrogenase isoenzymes are selected from the group consisting of FadE34/ChsE3 and CasC isoenzymes and combinations thereof.

7) The non-naturally occurring microbial organism of any of the foregoing claims derived from the genus Rhodococcus.

8) The non-naturally occurring microbial organism of any of the foregoing claims derived from Rhodococcus jostii RHA1.

9) The non-naturally occurring microbial organism of any of the foregoing claims derived from Rhodococcus sp. DSM 1444 or DSM 1445.

10) The non-naturally occurring microbial organism of claim 7, 8, or 9, having a disrupted expression sequence at:
a) RHA1_RS21845,
b) RHA1_RS28395,
c) RHA1_RS22120,
d) RHA1_RS12175,
e) RHA1_RS28370,
f) RHA1_RS40090,
g) or a combination thereof.

11) The non-naturally occurring microbial organism of claim 7, 8, or 9, having a disrupted expression sequence at:
a) RHA1_RS22090,
b) RHA1_RS12140,
c) RHA1_RS28305,
d) RHA1_RS28380,
e) RHA1_RS40180,
f) RHA1_RS40260,
g) or a combination thereof.

12) The non-naturally occurring microbial organism of any of claims 1-6 derived from the genus Mycobacterium.

13) The non-naturally occurring microbial organism of any of claims 1-6 derived from the genus Mycobacterium, comprising disrupted KstD enzymatic activity.

14) The non-naturally occurring microbial organism of any of claims 1-6 derived from the genus Mycobacterium, comprising disrupted Ksh enzymatic activity.

15) The non-naturally occurring microbial organism of any of claims 1-6 derived from the genus Mycobacterium, comprising disrupted KstD and Ksh enzymatic activity.

16) The non-naturally occurring microbial organism of any of claims 1-6 or 12-15, wherein the one or more acyl-CoA dehydrogenase isoenzymes are selected from the group consisting of ChsE1, ChsE2, ChsE3, and CasC isoenzymes and combinations thereof.

17) The non-naturally occurring microbial organism of any of claims 1-6 or 12-15, wherein the one or more acyl-CoA dehydrogenase isoenzymes comprise ChsE1, ChsE2, ChsE3, and CasC isoenzymes.

18) The non-naturally occurring microbial organism of any of claims 1-6 or 12-17, derived from Mycobacterium smegmatis MC(2) 155.

19) The non-naturally occurring microbial organism of claim 18 having a disrupted expression sequence at:
a) MSMEG_6041,
b) MSMEG_0603,
c) MSMEG_6039,
d) MSMEG_5941,
e) or a combination thereof.

20) The non-naturally occurring microbial organism of claim 18 having a disrupted expression sequence at:
a) MSMEG_6041,
b) MSMEG_0603,
c) MSMEG_6039, and
d) MSMEG_5941.

21) The non-naturally occurring microbial organism of any of claims 1-6 or 12-17 derived from the genus Mycobacterium neoaurum.

22) The non-naturally occurring microbial organism of any of claims 1-6 or 12-17 derived from the genus Mycobacterium neoaurum NRRL B-3805.

23) The non-naturally occurring microbial organism of any of claim 21 or 22, having a disrupted expression sequence at:
a) MyAD_RS24250;
b) MyAD_RS03655;
c) MyAD_RS24025;
d) MyAD_RS24020; or
e) a combination thereof.

24) The non-naturally occurring microbial organism of any of claim 21 or 22, having a disrupted expression sequence at:
a) MyAD_RS24250;
b) MyAD_RS03655;
c) MyAD_RS24025; and
d) MyAD_RS24020.

25) The organisms of any of claims 1-24, wherein the activity is disrupted by homologous recombination, mutagenesis, or a combination thereof.

26) A method of making a steroid comprising growing the non-naturally occurring microorganism of any of claims 1-25 on one or more phytosterols to produce KCEA and/or KCDA.

27) A method of making a steroid comprising growing the non-naturally occurring microorganism of any of claims 1-25 on β-sitosterol, campesterol, cholesterol, or a mixture thereof to produce KCEA and/or KCDA.

28) A method of making a steroid comprising growing the non-naturally occurring microorganism of any of claims 1-25 on β-sitosterol, campesterol, cholesterol, or a mixture thereof to produce KCEA and/or KCDA at a yield greater than 20%, 30%, 40%, 50%, 60%, 70%, or 80%.

29) The method of any of claims 26-28, wherein said growing occurs in the presence of a cyclodextrin.

30) The method of any of claims 26-29, further comprising converting said KCEA or KCDA to UDCA.


SEQUENCE LISTING (165 sequences)

SEQ ID NO: 1 - DNA, 42 bp, Artificial Sequence
  Primer
gagctcggtacccggggatccgaacgccttctcgtcggtgac

SEQ ID NO: 2 - DNA, 30 bp, Artificial Sequence
  Primer
gcaggatctgagcctggatcgaacccgctg

SEQ ID NO: 3 - DNA, 31 bp, Artificial Sequence
  Primer
gatccaggctcagatcctgctgacggtcgcg

SEQ ID NO: 4 - DNA, 38 bp, Artificial Sequence
  Primer
cagtgccaagcttgcatgcccgcgggcaggaacttgcg

SEQ ID NO: 5 - DNA, 24 bp, Artificial Sequence
  Prikmer
agcggataacaatttcacacagga

SEQ ID NO: 6 - DNA, 17 bp, Artificial Sequence
  Primer
gccgattcgcgacatcg

SEQ ID NO: 7 - DNA, 16 bp, Artificial Sequence
  Primer
gctacccgacgccgag

SEQ ID NO: 8 - DNA, 17 bp, Artificial Sequence
  Primer
gttttcccagtcacgac

SEQ ID NO: 9 - DNA, 41 bp, Artificial Sequence
  Primer
gagctcggtacccggggatcggcaacgccgtcatcgtcgac

SEQ ID NO: 10 - DNA, 24 bp, Artificial Sequence
  Primer
cgggcgagctcacccgtgcgcccg

SEQ ID NO: 11 - DNA, 27 bp, Artificial Sequence
  Primer
cgcacgggtgagctcgcccgcacgacc

SEQ ID NO: 12 - DNA, 37 bp, Artificial Sequence
  Primer
cagtgccaagcttgcatgccacgcgctcgaggttggc

SEQ ID NO: 13 - DNA, 15 bp, Artificial Sequence
  Primer
gcgcgaggatcgtcg

SEQ ID NO: 14 - DNA, 17 bp, Artificial Sequence
  Primer
ggccgagattccctgcc

SEQ ID NO: 15 - DNA, 40 bp, Artificial Sequence
  Primer
gagctcggtacccggggatcgcaacttcaggtggggccgg

SEQ ID NO: 16 - DNA, 26 bp, Artificial Sequence
  Primer
gctccgcggcacgcgcgaagcgggtc

SEQ ID NO: 17 - DNA, 26 bp, Artificial Sequence
  Primer
cttcgcgcgtgccgcggagcggtccg

SEQ ID NO: 18 - DNA, 38 bp, Artificial Sequence
  Primer
cagtgccaagcttgcatgccgacgcgggctgccagtgc

SEQ ID NO: 19 - DNA, 15 bp, Artificial Sequence
  Primer
gggtccgccgagcac

SEQ ID NO: 20 - DNA, 17 bp, Artificial Sequence
  Primer
gctctcaccgacgggtc

SEQ ID NO: 21 - DNA, 48 bp, Artificial Sequence
  Primer
gagctcggtacccggggatcacccactatttcgaaacgtacaaccggc

SEQ ID NO: 22 - DNA, 35 bp, Artificial Sequence
  Primer
tgtcggcaactcccacttcgatcatccggacttcg

SEQ ID NO: 23 - DNA, 32 bp, Artificial Sequence
  Primer
cgaagtgggagttgccgacaacctcgccaacg

SEQ ID NO: 24 - DNA, 38 bp, Artificial Sequence
  Primer
cagtgccaagcttgcatgcccacccgatggtgacgacc

SEQ ID NO: 25 - DNA, 17 bp, Artificial Sequence
  Primer
gccgtcctggacgtacg

SEQ ID NO: 26 - DNA, 16 bp, Artificial Sequence
  Primer
ggacgaacgcgttccg

SEQ ID NO: 27 - DNA, 39 bp, Artificial Sequence
  Primer
gagctcggtacccggggatcggcaacaccagcgcatcgc

SEQ ID NO: 28 - DNA, 29 bp, Artificial Sequence
  Primer
ggttctcttctgcgcccgcgtcgatttcg

SEQ ID NO: 29 - DNA, 35 bp, Artificial Sequence
  Primer
cgcgggcgcagaagagaacctcgcacgaaaggcgc

SEQ ID NO: 30 - DNA, 47 bp, Artificial Sequence
  Primer
cagtgccaagcttgcatgccggaagtatcgtgaccacgagttcggtc

SEQ ID NO: 31 - DNA, 17 bp, Artificial sequence
  Primer
ggtcccgaccaccatcg

SEQ ID NO: 32 - DNA, 18 bp, Artificial Sequence
  Primer
gcaccgaaatgctgcgac

SEQ ID NO: 33 - DNA, 46 bp, Artificial Sequence
  Primer
gagctcggtacccggggatctgtcgcgagtgatctctgcggtgtgg

SEQ ID NO: 34 - DNA, 31 bp, Artificial Sequence
  Primer
tggcatcgacggcaccggtgtcgatgactcg

SEQ ID NO: 35 - DNA, 30 bp, Artificial Sequence
  Primer
caccggtgccgtcgatgccaacctcgcggc

SEQ ID NO: 36 - DNA, 36 bp, Artificial Sequence
  Primer
cagtgccaagcttgcatgccagccgaccgccgtcac

SEQ ID NO: 37 - DNA, 21 bp, Artificial Sequence
  Primer
cgatgtcgaacctgatcaggc

SEQ ID NO: 38 - DNA, 20 bp, Artificial Sequence
  Primer
cgccattgccatccataacc

SEQ ID NO: 39 - DNA, 39 bp, Artificial Sequence
  Primer
gagctcggtacccggggatcactcgtcgatgttggcgcg

SEQ ID NO: 40 - DNA, 30 bp, Artificial Sequence
  Primer
tgccgtcagccattccaccggcaccgctac

SEQ ID NO: 41 - DNA, 34 bp, Artificial Sequence
  Primer
cggtggaatggctgacggcagcaccatcgagaac

SEQ ID NO: 42 - DNA, 39 bp, Artificial Sequence
  Primer
cagtgccaagcttgcatgccgtcccacgcgtccggaccc

SEQ ID NO: 43 - DNA, 15 bp, Artificial Sequence
  Primer
gccgagcacgaaccc

SEQ ID NO: 44 - DNA, 18 bp, Artificial Sequence
  Primer
ggcaactccacggactgg

SEQ ID NO: 45 - DNA, 42 bp, Artificial Sequence
  Primer
gagctcggtacccggggatcggatcgaatgtgatgcggtcgg

SEQ ID NO: 46 - DNA, 35 bp, Artificial Sequence
  Primer
ccatcacggagccgatcaccaggagatctacctcc

SEQ ID NO: 47 - DNA, 33 bp, Artificial Sequence
  Primer
ggtgatcggctccgtgatgggcaacgactacgc

SEQ ID NO: 48 - DNA, 39 bp, Artificial Sequence
  Primer
cagtgccaagcttgcatgccgacgtgatgcccgccgtgg

SEQ ID NO: 49 - DNA, 15 bp, Artificial Sequence
  Primer
acggcacgacgcatc

SEQ ID NO: 50 - DNA, 17 bp, Artificial Sequence
  Primer
gttgtcgcagacacccg

SEQ ID NO: 51 - DNA, 38 bp, Artificial Sequence
  Primer
gagctcggtacccggggatccgaggacccccggcagtg

SEQ ID NO: 52 - DNA, 36 bp, Artificial Sequence
  Primer
tgtagccgaagccgacgacgaggaaatcgactgtgg

SEQ ID NO: 53 - DNA, 34 bp, Artificial Sequence
  Primer
cgtcgtcggcttcggctacatcgccgcgctgcac

SEQ ID NO: 54 - DNA, 43 bp, Artificial Sequence
  Primer
cagtgccaagcttgcatgccggtgtccttcatgaacatcggcg

SEQ ID NO: 55 - DNA, 16 bp, Artificial Sequence
  Primer
gctcacagcaggcgac

SEQ ID NO: 56 - DNA, 17 bp, Artificial Sequence
  Primer
cgcgatctcctcctcgc

SEQ ID NO: 57 - DNA, 42 bp, Artificial Sequence
  Primer
gagctcggtacccggggatcggacgtgaactggtcgcccatg

SEQ ID NO: 58 - DNA, 30 bp, Artificial Sequence
  Primer
ccatcgcggtctgcgaagcggccgtgtagg

SEQ ID NO: 59 - DNA, 33 bp, Artificial Sequence
  Primer
cgcttcgcagaccgcgatggtcttctcctaccg

SEQ ID NO: 60 - DNA, 40 bp, Artificial Sequence
  Primer
cagtgccaagcttgcatgccgcactcagtgcactgcagcg

SEQ ID NO: 61 - DNA, 18 bp, Artificial Sequence
  Primer
ggcgtccagcatgatctg

SEQ ID NO: 62 - DNA, 21 bp, Artificial Sequence
  Primer
ggcaccgccagcaacgagatc

SEQ ID NO: 63 - DNA, 43 bp, Artificial Sequence
  Primer
gagctcggtacccggggatccggaaccgtaaacacggtagcgg

SEQ ID NO: 64 - DNA, 34 bp, Artificial Sequence
  Primer
cgataaatccgtcgactgtcagatcatgcgccgg

SEQ ID NO: 65 - DNA, 31 bp, Artificial Sequence
  Primer
gacagtcgacggatttatcgcggcacgtcac

SEQ ID NO: 66 - DNA, 42 bp, Artificial Sequence
  Primer
cagtgccaagcttgcatgccgaagtgcggtacgtccgatccg

SEQ ID NO: 67 - DNA, 20 bp, Artificial Sequence
  Primer
ggatgagtgagacccgatcc

SEQ ID NO: 68 - DNA, 17 bp, Artificial Sequence
  Primer
cccgcctgaatatgcgg

SEQ ID NO: 69 - DNA, 49 bp, Artificial Sequence
  Primer
gagctcggtacccggggatccaacgctggcatcaagctgggagaagcgg

SEQ ID NO: 70 - DNA, 36 bp, Artificial Sequence
  Primer
aggtggcaccgagtaggtcgaccttatgccgtcgag

SEQ ID NO: 71 - DNA, 30 bp, Artificial Sequence
  Primer
cgacctactcggtgccacctatccaggtgc

SEQ ID NO: 72 - DNA, 39 bp, Artificial Sequence
  Primer
cagtgccaagcttgcatgcccgccaaattccgccaccgg

SEQ ID NO: 73 - DNA, 19 bp, Artificial Sequence
  Primer
cgagtttcacagatcggcc

SEQ ID NO: 74 - DNA, 20 bp, Artificial Sequence
  Primer
agtgactactcatcgagcgg

SEQ ID NO: 75 - protein, 721 aa, Rhodococcus jostii RHA1
MTIATTEEQRAAAGSIQAWARSTDPLVTVRQGPADSWRTSWPSFASLGIFSVAVPEEVGGSGAEIVDLAAMLEQAATELVPGPVLSTALAALVVGRSSGAAAKRWSEDLADGAMPCAVALGSNSPVNATLGADGGLTVTGDAGFAIGGDTGVSVLLSADAGDGVVWCLIDGEADGLDVTVADTIDKSRPLARVRCDRVAVAADRVVTGVRDGLVADLAATLAAVEASGIAAWCLRTAVEYAKIREQFGKPIGSFQAIKHLCAEMLCRVEQSGAAAWDAAVAAEDAWDADGGELPIAAAVAASLTLDAAVETAKDCIQVLGGIGFTWEHDAHFYLRRATSLRQLLGGSARWRARVTELTRAGARRHLSIDLSGLDDERAQIRAEIADLVARPESERRAALAESGYLAPHWPAPYGRGARAAEQILIEEELAAAGVGRPDLVIGWWAVPTILEHGSAEQIERFATPTLRGEIIWCQLFSEPGAGSDLAALRTSAEKVDGGWRLNGQKVWTSRAQEADWAICLARTDRDAPKHRGISYFLVDMTSPGITISPLREITGDALFNEVYLEDVFVPDELVVGNLGDGWKLARTTLANERVAMGGGSSLGAAMEELLALAGVGDPVTDDRLGHLIGNALVGSQLELRTTLRQLDGQDPGPESSVRKLVGVRQRQAVAEFAMELGGVDGWVEGPLAREFLNTRCLSIAGGTTQILLTVAAERILGLPRG

SEQ ID NO: 76 - protein, 742 aa, Rhodococcus jostii RHA1
MTMGLTEEERDLRDSVRGWATRTVTPAVLREAVEAKTEQRPLFWKSLADLGVLGLHLPEDVGGAGCGLVELAVVTEELGRVLLPGPFLPTVLLGAVLHESGRTGELSGVADGSTLGAVALQPGSLRLVRDGGVVTLDGESGYVLGGQVGDLFLLAATDGDETVYVVVARDRLDVTDLPSHDVVRRSATAAANRLAIAEVDVLQIDSQRVRDLAATLFAAEASGVADWAVTTAADYANVRHQFGRPIGQFQGVKHRVARMLCLAEQARVCAWDAARAMNPGSSARAATAVPRCSAPEASLAAAVAGATAPDAAFAVTKDCIQVLGGIGYTWEHDAHLYLRRAQSLRILLGSTASWRRRVARATLGGARRVLGIDLPSDAEDIRADVVAELAPAAAMGEAERRTYLAEKGYTAPHLPTPWGRGAGAVEQLVIAEELRAAALKPHDMIIGNWVVPTLIAHGSEEQLQRFVPPSLRGDLVWCQLFSEPGAGSDLAGLTTRATKVDGGWVLQGQKVWTSMARDADWGICLARTDGSVPKHKGLSYFLIDMSGSDGLDIRPLREITGEALFNEVFLDDVFVPDELLVGEPGDGWKLARTTLANERVSLSHDSSLGSGGEALLTLAGELPGGIDEEQLTVLGKVLCDAQSGGLMGLRTTLRTIAGGQPGAESSVAKLLGVEHIQQVWEVAMDWAGPRALLGDQPRTSATQMFLNSQCMSIAGGTTNVQLNIIGERLLGLPRDPEPGKGA

SEQ ID NO: 77 - DNA, 39 bp, Artificial Sequence
  Primer
gagctcggtacccggggatctgccgcaggatgtcgaccg

SEQ ID NO: 78 - DNA, 28 bp, Artificial Sequence
  Primer
ggatctgctcgcggaccatatcgcgggc

SEQ ID NO: 79 - DNA, 32 bp, Artificial Sequence
  Primer
tatggtccgcgagcagatcctgctgacgttgg

SEQ ID NO: 80 - DNA, 42 bp, Artificial Sequence
  Primer
cagtgccaagcttgcatgccgcgcatccacgtcctggcgccc

SEQ ID NO: 81 - DNA, 17 bp, Artificial Sequence
  Primer
gtcggcgaacacgaacg

SEQ ID NO: 82 - DNA, 16 bp, Artificial Sequence
  Primer
gcggcctccgattcgg

SEQ ID NO: 83 - DNA, 41 bp, Artificial Sequence
  Primer
gagctcggtacccggggatcggatggtggacccgacgatcg

SEQ ID NO: 84 - DNA, 34 bp, Artificial Sequence
  Primer
cggtggtgccgaacgacttgaccgaatcggccag

SEQ ID NO: 85 - DNA, 31 bp, Artificial Sequence
  Primer
caagtcgttcggcaccaccgaggtgcagttg

SEQ ID NO: 86 - DNA, 40 bp, Artificial Sequence
  Primer
cagtgccaagcttgcatgcctaccgcatcgcgtgtcgacg

SEQ ID NO: 87 - DNA, 16 bp, Artificial Sequence
  Primer
cggcaacgtcgccaag

SEQ ID NO: 88 - DNA, 19 bp, Artificial Sequence
  Primer
gccgacgaacatgatggtg

SEQ ID NO: 89 - DNA, 44 bp, Artificial Sequence
  Primer
gagctcggtacccggggatcggcagtcaccatcagaccatcgtg

SEQ ID NO: 90 - DNA, 32 bp, Artificial Sequence
  Primer
cctggcaaccgtaccgaagccggtccgcggcg

SEQ ID NO: 91 - DNA, 29 bp, Artificial Sequence
  Primer
gcttcggtacggttgccaggcgacacccg

SEQ ID NO: 92 - DNA, 40 bp, Artificial Sequence
  Primer
cagtgccaagcttgcatgccggcgtcggcgaacacgaacg

SEQ ID NO: 93 - DNA, 18 bp, Artificial Sequence
  Primer
gccgttgacgatgtagcc

SEQ ID NO: 94 - DNA, 16 bp, Artificial Sequence
  Primer
ccgtttgccgcgtcag

SEQ ID NO: 95 - DNA, 38 bp, Artificial Sequence
  Primer
gagctcggtacccggggatctgttctgcacgccggcgc

SEQ ID NO: 96 - DNA, 27 bp, Artificial Sequence
  Primer
tcatggcgggcatgccggcagcaccgc

SEQ ID NO: 97 - DNA, 34 bp, Artificial Sequence
  Primer
tgccggcatgcccgccatgacattcggttacctg

SEQ ID NO: 98 - DNA, 40 bp, Artificial Sequence
  Primer
cagtgccaagcttgcatgcccccgtcgtgtcgaacggacc

SEQ ID NO: 99 - DNA, 20 bp, Artificial Sequence
  Primer
cgtcgatgttgcgaatgtcg

SEQ ID NO: 100 - DNA, 16 bp, Artificial Sequence
  Primer
ccttggcgaaggtcgg

SEQ ID NO: 101 - protein, 572 aa, Rhodococcus jostii RHA1
MSKHEYDIVVVGSGAGGMTAALTAAHKGLSVVILEKAPHFGGSTARSGGGVWIPNNEALIAAGVEDTTDAARTYLHSIIGDVVPKDRIDAYIDRGPEMLSFVLKNSPLKLQWVPEYSDYYPEAPGGRLGGRSVEPTPFDGRKLGADLAKLEPDYVKAPSNFVITQADYRWLNLLMRNPRGPLRAARVGLRFLGAKLTGKRLLVRGQALVAGLQAGLKAAGVPILLDTPLTDLYVEDGVVKGVNATVDGQPQVFRARRGVVIASGGFEHNDEARKKYQRAPIGTEWTVGAKANTGDGIWAGEKLGAALDLMEDAWWGPSIPLTGGPWFALSERSLPGCIMINGSGKRFVNESAPYVEATHAMYGGTHGQGDGPGENVPCWLVLDQRYRDRYAFAGVTPRSPFPGRWLKAGVIVKAGTVAELAGKIGVPVDALTETVSRFNTFARNGKDEDFGRGESGYDHYYGDPRNKPNPSLGALEKAPFFAVKMVPGDLGTKGGLRTDRNARVLRADGSTIENLYAVGNASGPVMGHTYAGPGATIGPAMTFGYLAVLDILDKSSQADSPTSLTGDTTNAH

SEQ ID NO: 102 - DNA, 37 bp, Artificial Sequence
  Primer
gcggcaaactagttgtatcggtcgggacggacagcac

SEQ ID NO: 103 - DNA, 38 bp, Artificial Sequence
  Primer
cgatacaactagtttgccgcgctagtcaggtaatcggc

SEQ ID NO: 104 - DNA, 46 bp, Artificial Sequence
  Primer
cgagctcggtacccggggatcgatggtctcgtagggaacctggcgc

SEQ ID NO: 105 - DNA, 21 bp, Artificial Sequence
  Primer
tcggcaccgtggctaccgacc

SEQ ID NO: 106 - DNA, 24 bp, Artificial Sequence
  Primer
tccaggatgcgcttacgtcgttcc

SEQ ID NO: 107 - DNA, 27 bp, Artificial Sequence
  Primer
tgatccactggctggagaccgtacagg

SEQ ID NO: 108 - DNA, 53 bp, Artificial Sequence
  Primer
aacgacggccagtgccaagctggtttcgagagcagcttcctactcgatcatgc

SEQ ID NO: 109 - DNA, 42 bp, Artificial Sequence
  Primer
gacgttcactagtgtcgttgtgttcagagttgatcgcgatgg

SEQ ID NO: 110 - DNA, 34 bp, Artificial Sequence
  Primer
caacgacactagtgaacgtctgctgggcctgccc

SEQ ID NO: 111 - DNA, 44 bp, Artificial Sequence
  Primer
cgagctcggtacccggggatcggtgccgttcaagtcgaacgagg

SEQ ID NO: 112 - DNA, 25 bp, Artificial Sequence
  Primer
cagcgagcaggccgataccatttcc

SEQ ID NO: 113 - DNA, 24 bp, Artificial Sequence
  Primer
cctggtacaccgcggacacgatgc

SEQ ID NO: 114 - DNA, 24 bp, Artificial Sequence
  Primer
acaacctcgcgcgctacaaggtcc

SEQ ID NO: 115 - DNA, 42 bp, Artificial Sequence
  Primer
aacgacggccagtgccaagcttcacctcgatcaccggcacgc

SEQ ID NO: 116 - DNA, 46 bp, Artificial Sequence
  Primer
cttcgatactagttaccaccactacgtcaaagacactgtagtcctg

SEQ ID NO: 117 - DNA, 36 bp, Artificial Sequence
  Primer
ggtggtaactagtatcgaaggcctctatgcggcagg

SEQ ID NO: 118 - DNA, 44 bp, Artificial Sequence
  Primer
cgagctcggtacccggggatcccgtgtcgatacgcagaccgtgg

SEQ ID NO: 119 - DNA, 21 bp, Artificial Sequence
  Primer
ccagatgcgcccgcagattcg

SEQ ID NO: 120 - DNA, 26 bp, Artificial Sequence
  Primer
ggccaacacatcatcctcggtacacc

SEQ ID NO: 121 - DNA, 23 bp, Artificial Sequence
  Primer
ccagaaccgttagcgcgagtgtg

SEQ ID NO: 122 - DNA, 1897 bp, Bacillus subtilis
gatccatggcccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgccatggaagc

SEQ ID NO: 123 - DNA, 40 bp, Artificial Sequence
  Primer
tcgcgcgcggccgcggatccgctgaacgccttttcgtcgg

SEQ ID NO: 124 - DNA, 35 bp, Artificial Sequence
  Primer
gaatctgactagtggcctctatcgattcctggacc

SEQ ID NO: 125 - DNA, 36 bp, Artificial Sequence
  Primer
cgcgactagtctcaacacgcgatgcctgtcgattgc

SEQ ID NO: 126 - DNA, 30 bp, Artificial Sequence
  Primer
cgcgaagcttgcacttcaccggcgaggatc

SEQ ID NO: 127 - DNA, 17 bp, Artificial Sequence
  Primer
agaacgtcgcgtgagcc

SEQ ID NO: 128 - DNA, 18 bp, Artificial Sequence
  Primer
gccggcaccgagaagttc

SEQ ID NO: 129 - DNA, 26 bp, Artificial Sequence
  Primer
gctgccgaccttgattccgatatccg

SEQ ID NO: 130 - DNA, 25 bp, Artificial Sequence
  Primer
tgacgacgtactcgtcaccgtttcg

SEQ ID NO: 131 - DNA, 40 bp, Artificial Sequence; identical to SEQ ID NO: 15
  Primer

SEQ ID NO: 132 - DNA, 36 bp, Artificial Sequence
  Primer
acgtggagcggatcactagtacgcgcgaagcgggtc

SEQ ID NO: 133 - DNA, 36 bp, Artificial Sequence
  Primer
gatggagctgcacaactagtgccgcggagcggtccg

SEQ ID NO: 134 - DNA, 37 bp, Artificial Sequence
  Primer
ttacgccaagcttgcatgccgacgcgggctgccagtg

SEQ ID NO: 135 - DNA, 23 bp, Artificial Sequence
  Primer
gcagcacgatcgtggtgtcgttg

SEQ ID NO: 136 - DNA, 21 bp, Artificial Sequence
  Primer
atggcgcattcgatcgcaccg

SEQ ID NO: 137 - DNA, 41 bp, Artificial Sequence
  Primer
acgtggagcggatcactagtagcctggatcgaacccgctgc

SEQ ID NO: 138 - DNA, 40 bp, Artificial Sequence
  Primer
gatggagctgcacaactagtcagatcctgctgacggtcgc

SEQ ID NO: 139 - DNA, 25 bp, Artificial Sequence
  Primer
acacccggaattcggcgagactctc

SEQ ID NO: 140 - DNA, 32 bp, Artificial Sequence
  Primer
tggtgaagatcttctgcccgttgacgatgtac

SEQ ID NO: 141 - DNA, 39 bp, Artificial Sequence
  Primer
acgtggagcggatcactagtgagctcacccgtgcgcccg

SEQ ID NO: 142 - DNA, 38 bp, Artificial Sequence
  Primer
gatggagctgcacaactagtaagctcgcccgcacgacc

SEQ ID NO: 143 - DNA, 25 bp, Artificial Sequence
  Primer
gccagaagaacccaaggagcggaac

SEQ ID NO: 144 - DNA, 24 bp, Artificial Sequence
  Primer
cccggcgatgctgttcagctcttc

SEQ ID NO: 145 - DNA, 48 bp, Artificial Sequence
  Primer
acgacggccagtgccaagcttgatctgcatgtcctccagccggtttcg

SEQ ID NO: 146 - DNA, 37 bp, Artificial Sequence; identical to SEQ ID NO: 102
  Primer

SEQ ID NO: 147 - DNA, 38 bp, Artificial Sequence; identical to SEQ ID NO: 103
  Primer

SEQ ID NO: 148 - DNA, 46 bp, Artificial Sequence
  Primer
atcgcgcgcggccgcggatccgatggtctcgtagggaacctggcgc

SEQ ID NO: 149 - DNA, 26 bp, Artificial Sequence
  Primer
tcgggctgcagcagtttgaagaagcc

SEQ ID NO: 150 - DNA, 21 bp, Artificial Sequence
  Primer
cagcagcgccggataatgcgc

SEQ ID NO: 151 - DNA, 53 bp, Artificial Sequence; identical to SEQ ID NO: 108
  Primer

SEQ ID NO: 152 - DNA, 42 bp, Artificial Sequence; identical to SEQ ID NO: 109
  Primer

SEQ ID NO: 153 - DNA, 34 bp, Artificial Sequence; identical to SEQ ID NO: 110
  Primer

SEQ ID NO: 154 - DNA, 47 bp, Artificial Sequence
  Primer
tatcgcgcgcggccgcggatcctcggtgccgttcaagtcgaacgagg

SEQ ID NO: 155 - DNA, 26 bp, Artificial Sequence
  Primer
cgacttctccgagcactatctcgcgg

SEQ ID NO: 156 - DNA, 29 bp, Artificial Sequence
  Primer
aatcgtcgatcatcattttgaccagcggc

SEQ ID NO: 157 - DNA, 45 bp, Artificial Sequence
  Primer
actctagaggatccgcggccgccgcgatgccgatgtcgatcgcac

SEQ ID NO: 158 - DNA, 35 bp, Artificial Sequence
  Primer
tttcgcgactagtctcgggcttcggcgtgaagtcc

SEQ ID NO: 159 - DNA, 35 bp, Artificial Sequence
  Primer
gcccgagactagtcgcgaaatgatcgcggcgtcgg

SEQ ID NO: 160 - DNA, 48 bp, Artificial Sequence
  Primer
attcgatatcgcgcgcggccgccggcactggtggcgccgtattcgtgc

SEQ ID NO: 161 - DNA, 21 bp, Artificial Sequence
  Primer
gctactggatcgtcaccaagc

SEQ ID NO: 162 - DNA, 19 bp, Artificial Sequence
  Primer
ccaacttcgacgacgaggc

SEQ ID NO: 163 - DNA, 22 bp, Artificial Sequence
  Primer
gcgtgacaccaagttcttctgg

SEQ ID NO: 164 - DNA, 50 bp, Artificial Sequence
  Primer
aacgacggccagtgccaagctcctcgaactgattgggcatgtcgatgtcc

SEQ ID NO: 165 - DNA, 44 bp, Artificial Sequence
  Primer
tatcgcgcgcggccgcggatccggttggttgcggcgtgtttgcg